(19) United States
(12) Patent Application Publication
(10) Pub. No.: US 2026/0240689 A1
(43) Pub. Date: Aug. 20, 2026

(54) ABSORBENT ARTICLES AND METHODS FOR APPLYING A WAIST PANEL TO ELASTICIZE A WAIST REGION WITH INTEGRALLY FORMED SIDE PANELS

(71) Applicant: The Procter & Gamble Company, Cincinnati, OH (US)

(72) Inventors: Uwe SCHNEIDER, Cincinnati, OH (US); Jeromy Thomas RAYCHECK, South Lebanon, OH (US)

(21) Appl. No.: 19/536,592

(22) Filed: Feb. 11, 2026

Related U.S. Application Data
(60) Provisional application No. 63/760,680, filed on Feb. 20, 2025.

(51) Int. Cl. A61F 13/15; A61F 13/49; A61F 13/56

(52) U.S. Cl. CPC A61F 13/15593; A61F 13/15739; A61F 13/4902; A61F 13/565; A61F 2013/49025

(57) ABSTRACT

A method for assembling absorbent articles is presented. The method includes steps of providing a secondary carrier substrate including fasteners intermittently spaced apart from each other along a length of the secondary carrier substrate; separating the secondary carrier substrate into discrete fastener panels, each fastener panel including a first surface and an opposing second surface, wherein at least one fastener is positioned on the first surface; providing an elastic laminate including a first surface and an opposing second surface; bonding second surfaces of the fastener panels with the first surface of the elastic laminate to form a waist panel laminate; advancing a primary carrier substrate in a machine direction; and bonding the waist panels with the primary carrier substrate.

### CROSS REFERENCE TO RELATED APPLICATION

This application claims the benefit, under 35 U.S.C. §119(e), of U.S. Provisional Application No. 63/760,680, filed February 20, 2025, the entire disclosure of which is incorporated herein by reference.

### FIELD

The present disclosure relates to absorbent articles with waist regions including integrally formed side panels, and more particularly, absorbent articles and methods for making absorbent articles with a waist panel to elasticize a central portion of the waist region as well as the integrally formed side panels.

### BACKGROUND

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from other advancing webs. Webs of material and component parts used to manufacture diapers may include: backsheets, topsheets, leg cuffs, waistbands, absorbent core components, front and/or back ears, fastening components, and various types of elastic webs and parts such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

Some absorbent articles, such as diapers, have components that include waist panels, which may also be referred to as waistbands. In some configurations, waistbands may be provided as a single layer of elastic material, such as an elastic film. In some configurations, the waistbands may be provided as an elastic laminate that may include elastic material bonded to one or more substrates such as nonwovens, wherein the elastic material may include an elastic film and/or elastic strands. In some assembly operations, the waistbands are joined to an advancing carrier web, such as a continuous topsheet or backsheet web, while the waistbands are in a stretched condition. As such, when the waistbands relax, the carrier web gathers to form corrugations. The resulting laminate is stretchable to the extent that the corrugations allow the waistband to elongate.

In some configurations, waist panels may be bonded with diapers in a rear waist region to provide a stretch characteristic to portions of the rear waist region. In addition, discrete side panels with fastener components may be connected with laterally outer regions of the rear waist region. In some configurations, it may also be desirable to provide elastic side panels, which may operate in conjunction with the elastic waist panel to provide elasticity to the rear waist region. However, combining such elastic side panels and waist panels to a diaper may increase the complexity of the manufacturing process. Such complexities may be a result of a need to combine and otherwise handle a plurality of discrete elastic components while maintaining a stretched state of such components. The aforementioned challenges may be exacerbated in absorbent article assembly processes operating at relatively high speed production rates. In addition, the aforementioned assemblage of discrete elastic components may result in gaps or breaks in the continuity of elasticity in the rear waist region between such components.

In an attempt to reduce complexities associated with assembling and handling discrete side panels, some diapers may be configured with integrally formed side panels. However, assembly and manufacturing challenges remain when it is desirable to elasticize such integral side panels to provide elastic characteristics to a waist region in conjunction with a separately added elastic waist panel.

Consequently, it would be beneficial to provide assembly processes and diapers with waist panels applied to a waist region with integrally formed side panels to as to provide elasticity to a central portion of the waist region as well as the integrally formed side panels without gaps or breaks in the continuity of elasticity.

### SUMMARY

In one form, a method for assembling absorbent articles comprises steps of: providing a secondary carrier substrate comprising fasteners intermittently spaced apart from each other along a length of the secondary carrier substrate; separating the secondary carrier substrate into discrete fastener panels, each fastener panel comprising a first surface and an opposing second surface, wherein at least one fastener is positioned on the first surface; providing an elastic laminate comprising a first surface and an opposing second surface; bonding second surfaces of the fastener panels with the first surface of the elastic laminate to form a waist panel laminate, the fastener panels being intermittently spaced from each other along a length of the elastic laminate; separating the waist panel laminate along at least one line of separation extending through the elastic laminate and the fastener panels to form discrete waist panels, each discrete waist panel comprising a first surface and an opposing second surface, a first end region, a second end region, and central region separating the first end region from the second end region, wherein each waist panel comprises a first portion of a first separated fastener panel on the first surface of the first end region and a second portion of a second separated fastener panel on the first surface of the second end region; advancing a primary carrier substrate in a machine direction, the primary carrier substrate comprising a first edge separated from a second edge in a cross direction, wherein first and second edges of the primary carrier substrate are contoured to define relatively narrow regions arranged intermittently between relatively wide regions along the machine direction, wherein the relatively wide regions comprise integrally formed wing panels; and bonding the waist panels with the primary carrier substrate, wherein the waist panels extend in a cross direction such that the first end region extends outboard from the first edge and the second end region extends outboard from the second edge of the primary carrier substrate. wherein the waist panels extend in the cross direction such that the first end region extends outboard from the first edge and the second end region extends outboard from the second edge of the wing panels of the primary carrier substrate.

### BRIEF DESCRIPTION OF THE DRAWINGS

FIG. 1A is a partially cut away plan view of an absorbent article in the form of a taped diaper having integrally formed sides panels and including a waist panel bonded with a wearer facing surface that may be assembled in accordance with the present disclosure with the portion of the diaper that faces away from a wearer oriented towards the viewer.

FIG. 1B is a plan view of the absorbent article of FIG. 1A with the portion of the diaper that faces toward a wearer oriented towards the viewer.

FIG. 1C is a partially cut away plan view of an absorbent article in the form of a taped diaper having integrally formed sides panels and including a waist panel bonded with a garment facing surface that may be assembled in accordance with the present disclosure with the portion of the diaper that faces away from a wearer oriented towards the viewer.

FIG. 1D is a plan view of the absorbent article of FIG. 1A with the portion of the diaper that faces toward a wearer oriented towards the viewer.

FIG. 2 is a detailed view of a waist panel positioned on a topsheet with the portion of the diaper that faces toward a wearer oriented towards the viewer.

FIG. 3 is a detailed view of a waist panel positioned on a backsheet with the portion of the diaper that faces toward a wearer oriented towards the viewer.

FIG. 4 is a schematic side view of an apparatus for assembling absorbent articles with integrally formed sides panels and including a waist panel bonded with a waist region to elasticize a central portion and the side panels in the waist region.

FIG. 5A is a view of a primary carrier substrate taken along section 5A-5A in FIG. 4.

FIG. 5B is a view of the primary carrier substrate taken along section 5B-5B in FIG. 4.

FIG. 5C is a view of a continuous length of absorbent articles with waist panels taken along section 5C-5C in FIG. 4.

FIG. 5D is a view of discrete absorbent articles with waist panels taken along section 5D-5D in FIG. 4.

FIG. 6A is a view of a continuous waist panel laminate taken along section 6A-6A in FIG. 4.

FIG. 6B is a view of discrete waist panels advancing on a cutting device taken along section 6B-6B in FIG. 4.

FIG. 6C is a view of a discrete waist panel advancing and being reoriented on a transfer device taken along section 6C-6C in FIG. 4.

FIG. 7A is a view of a continuous elastic laminate taken along section 7A-7A in FIG. 4.

FIG. 7A1 is a view of the continuous length of the elastic laminate taken along section 7A1-7A1 in FIG. 7A.

FIG. 7B is a view of the continuous elastic laminate taken along section 7B-7B in FIG. 4.

FIG. 7C is a view of a continuous length of fastener panels taken along section 7C-7C in FIG. 4.

FIG. 7D is a view of discrete fastener panels advancing in a machine direction taken along section 7D-7D in FIG. 4.

FIG. 8A is a view of a secondary carrier substrate taken along section 8A-8A in FIG. 4.

FIG. 8B is a view of continuous length of fastener material taken along section 8B-8B in FIG. 4.

FIG. 8B1 is a view of the continuous length of fastener material taken along section 8B1-8B1 in FIG. 8B.

FIG. 8C is a view of discrete fasteners advancing in a machine direction taken along section 8C-8C in FIG. 4.

### DETAILED DESCRIPTION

The following term explanations may be useful in understanding the present disclosure:

“Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

“Relaxed state” defines a length of material when not stretched by an applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern. It is to be appreciated that nonwovens having various basis weights can be used in accordance with the methods herein.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Patent Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, which are all incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Patent Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, which are all incorporated by reference herein.

Aspects of the present disclosure relate to methods for assembling absorbent articles with integrally formed side panels and a waist panel that elasticizes a central portion of the waist region as well as the integrally formed side panels. As discussed in more detail below, methods for assembling absorbent articles may include providing a secondary carrier substrate comprising fasteners intermittently spaced apart from each other along a length of the secondary carrier substrate. The secondary carrier substrate may then be separated into discrete fastener panels, each fastener panel comprising a first surface and an opposing second surface, and at least one fastener may be positioned on the first surface. The second surface of the fastener panel may be bonded with a first surface of an elastic laminate to form a waist panel laminate with the fastener panels being intermittently spaced from each other along a length of the elastic laminate. The waist panel laminate may be separated along at least one line of separation extending through the elastic laminate and the fastener panels to form discrete waist panels. Each discrete waist panel may comprise a first surface and an opposing second surface, a first end region, a second end region, and central region separating the first end region from the second end region. In addition, each waist panel may comprise a first portion of a first separated fastener panel on the first surface of the first end region and a second portion of a second separated fastener panel on the first surface of the second end region. The waist panels may be bonded with a primary carrier substrate comprising a first edge separated from a second edge in a cross direction. The first and second edges of the primary carrier substrate may be contoured to define relatively narrow regions arranged intermittently between relatively wide regions along the machine direction, wherein the relatively wide regions comprise integrally formed wing panels. In turn, each waist panel may extend in a cross direction in a stretched state such that the first end region extends outboard from the first edge and the second end region extends outboard from the second edge of the wing panels of the primary carrier substrate. In some configurations, the primary carrier substrate may comprise a continuous topsheet, and the second surfaces of the waist panels may be bonded with the continuous topsheet substrate in a stretched state. In some configurations, the primary carrier substrate may comprise a continuous backsheet, and the first surfaces of the waist panels may be bonded with the continuous backsheet substrate in a stretched state.

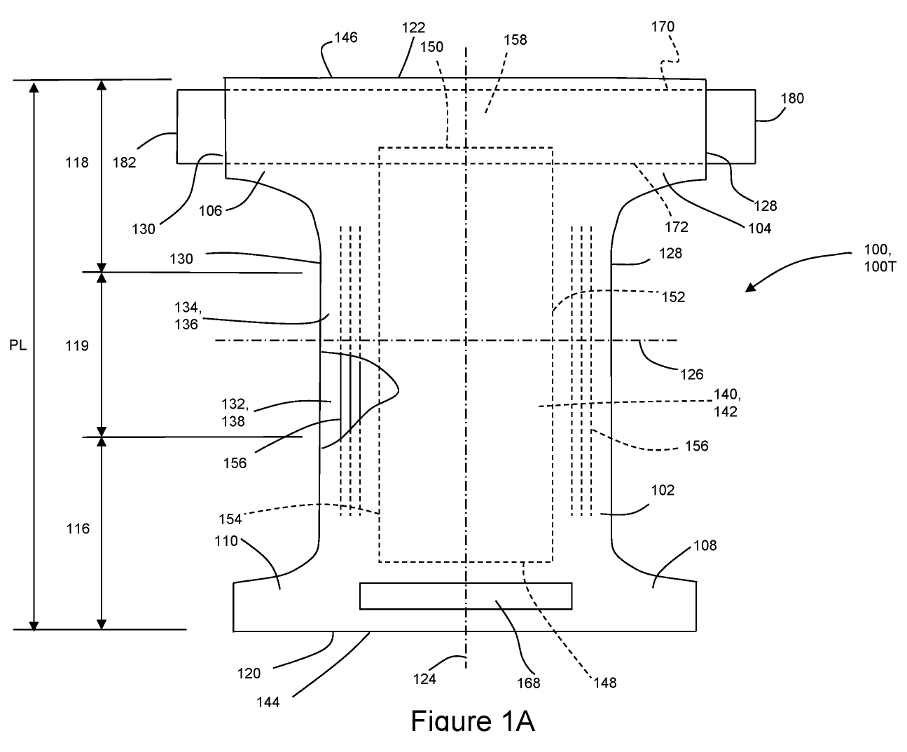

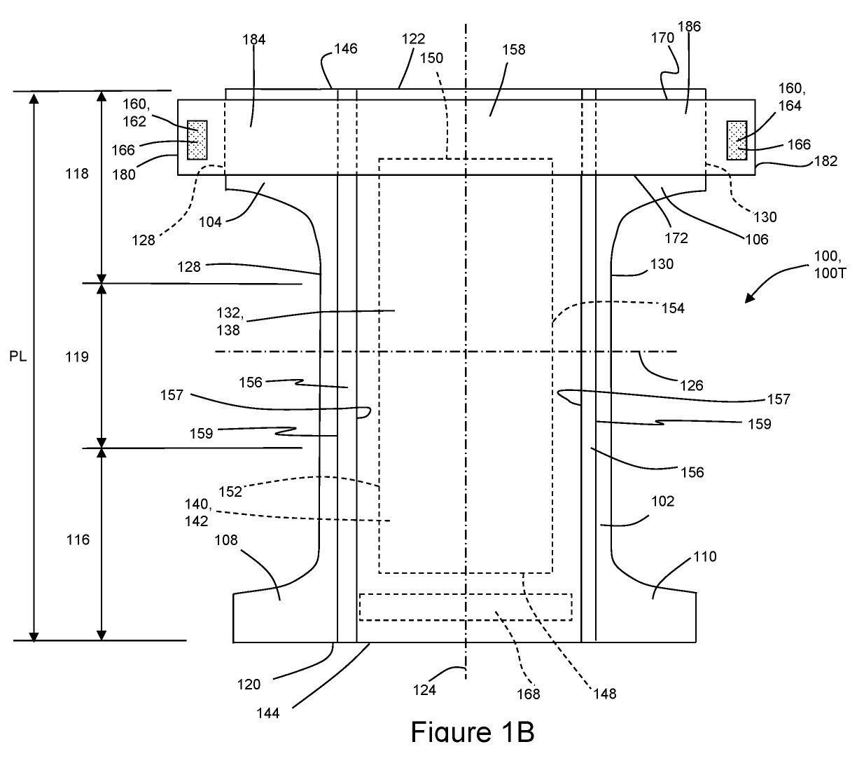

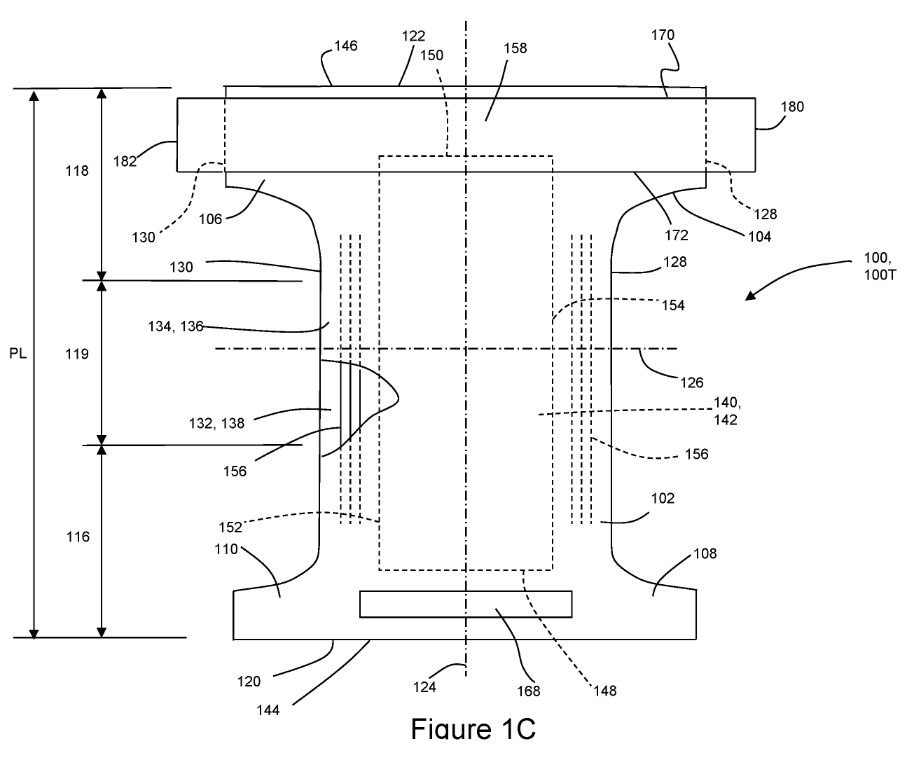

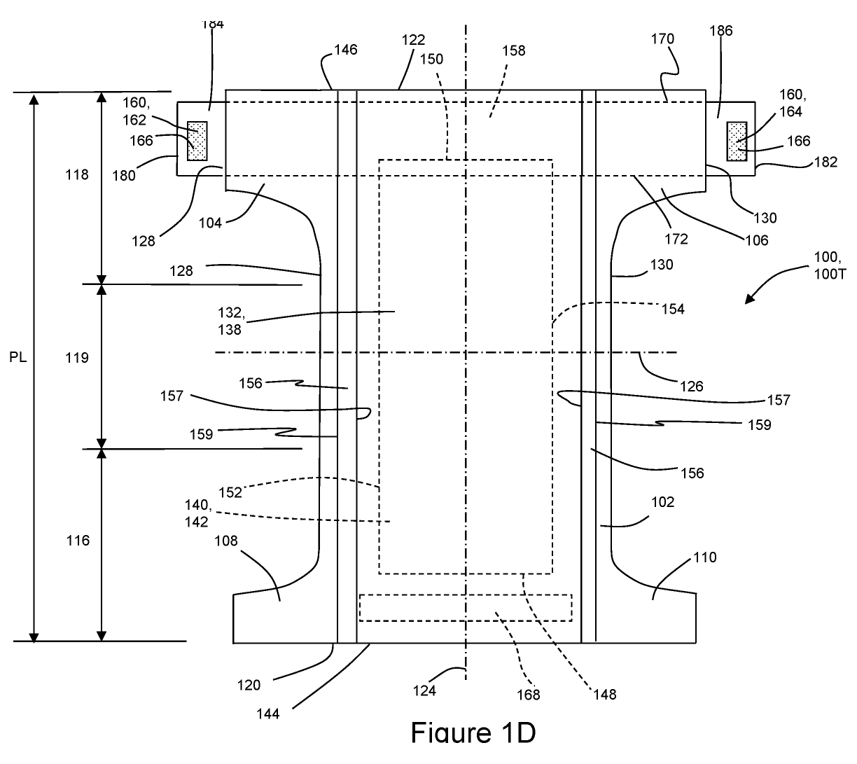

For the purposes of a specific illustration, FIGS. 1A and 1B show an example of an absorbent article 100 that may be assembled in accordance with the present disclosure. In particular, FIG. 1A shows one example of a plan view of an absorbent article 100 configured as a taped diaper 100T, with the portion of the diaper that faces away from a wearer oriented towards the viewer. And FIG. 1B shows a plan view of the diaper 100 with the portion of the diaper that faces toward a wearer oriented towards the viewer. The taped diaper 100T shown in FIGS. 1A and 1B includes an absorbent chassis 102, first and second rear side panels 104 and 106; and first and second front side panels 108 and 110. Although much of the following discussion is made with reference to an example taped diaper configuration, it is to be appreciated that the component configurations and methods herein may be adapted for application to diaper pant configurations.

As shown in FIGS. 1A and 1B, the absorbent article 100 and the chassis 102 each include a first waist region 116, a second waist region 118, and a crotch region 119 disposed intermediate the first and second waist regions. The first waist region 116 may be configured as a front waist region, and the second waist region 118 may be configured as a back waist region. In some configurations, the length of each of the front waist region, back waist region, and crotch region may be 1/3 of the length of the absorbent article 100. The absorbent article 100 may also include a laterally extending first waist edge 120 in the first waist region 116, wherein the first waist edge 120 may be configured as a front waist edge. In addition, the absorbent article 100 may include a laterally extending second waist edge 122 in the second waist region 118, wherein the second waist edge 122 may be configured as a back waist edge. To provide a frame of reference for the present discussion, the diaper 100T in FIGS. 1A and 1B is shown with a longitudinal axis 124 and a lateral axis 126. The longitudinal axis 124 may extend through a midpoint of the front waist edge 120 and through a midpoint of the back waist edge 122. And the lateral axis 126 may extend through a midpoint of a first longitudinal or right side edge 128 and through a midpoint of a second longitudinal or left side edge 130.

As shown in FIGS. 1A and 1B, the absorbent article 100 includes an inner, wearer facing surface 132, and an outer, garment facing surface 134. As such, it is also to be appreciated that the various components of the absorbent article described below may each include inner, wearer facing surfaces 132, and an outer, garment facing surfaces 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the absorbent article 100 may also include other features, such as leg gasketing elements, waist panels, and/or flaps, e.g., side panels and/or ears, to enhance the fits around the legs and waist of the wearer, to enhance the fit around the legs of the wearer.

As shown in FIGS. 1A and 1B, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 1A, the laterally extending end edges 144 and 146 may form a portion of the laterally extending front waist edge 120 in the front waist region 116 and a portion of the longitudinally opposing and laterally extending back waist edge 122 in the back waist region 118. The distance between the first lateral end edge 144 and the second lateral end edge 146 may define a pitch length, PL, of the chassis 102. When the absorbent article 100 is worn on the lower torso of a wearer, the front waist edge 120 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As shown in FIGS. 1A and 1B, the first and second rear side panels 104, 106 and/or the first and second front side panels 108, 110 may be integrally formed regions of the chassis 102. For example, the first longitudinal side edge 128 and the second longitudinal side edge 130 may be contoured such that the topsheet 138 and backsheet 136 are formed with relatively narrow crotch regions 119 and relatively wide first and/or second waist regions 116, 118 so as to define the shapes of the first and second rear side panels 104, 106 and/or the first and second front side panels 108, 110. It is to be appreciated that in some configurations, the first and second rear side panels 104, 106 and/or the first and second front side panels 108, 110 may comprise separate components that are bonded with the chassis 102.

As previously mentioned, the absorbent article 100 may include a backsheet 136. The backsheet 136 may also define the outer surface 134 of the chassis 102. The backsheet 136 may be impervious to fluids (e.g., menses, urine, and/or runny feces) and may be manufactured in part from a thin plastic film, although other flexible liquid impervious materials may also be used. The backsheet 136 may prevent the exudates absorbed and contained in the absorbent core from wetting articles which contact the absorbent article 100, such as bedsheets, pajamas and undergarments. The backsheet 136 may also comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet 136 may also comprise an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the absorbent article 100.

Also described above, the absorbent article 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer’s skin. Exemplary apertured films may include those described in U.S. Patent Nos. 5,628,097; 5,916,661; 6,545,197; and 6,107,539, which are all incorporated by reference herein.

As mentioned above, the absorbent article 100 may also include an absorbent assembly 140. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the absorbent article. Exemplary absorbent structures for use as the absorbent core of the present disclosure are described in U.S. Patent Nos. 4,610,678; 4,673,402; 4,888,231; and 4,834,735, which are all incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Patent Nos. 5,599,335; 5,562,646; 5,669,894; and 6,790,798 as well as U.S. Patent Publication Nos. 2004/0158212 A1 and 2004/0097895 A1, which are all incorporated by reference herein.

In some configurations, the absorbent assembly 140 may include an acquisition system disposed between the topsheet 138 and a wearer facing side of the absorbent core 142. The acquisition system may be in direct contact with the absorbent core 142 and may comprise a single layer or multiple layers, such as an upper acquisition layer (also referred to herein as a first acquisition layer) facing towards the wearer's skin and a lower acquisition layer (also referred to herein as a second acquisition layer) facing the garment of the wearer. In some configurations, the acquisition system may function to receive a surge of liquid, such as a gush of urine. As such, the acquisition system may serve as a temporary reservoir for liquid until the absorbent core 142 can absorb the liquid. Exemplary acquisition systems and associated manufacturing processes are described in U.S. Patent Nos. 8,603,277 and 8,568,566; U.S. Patent Publication Nos. 2012/0316046 A1 and 2014/0163504 A1, all of which are hereby incorporated by reference herein.

In some configurations, the acquisition system may include chemically cross-linked cellulosic fibers. Such cross-linked cellulosic fibers may have various absorbency properties. Exemplary chemically cross-linked cellulosic fibers are disclosed in U.S. Patent No. 5,137,537. Citric acid is an exemplary cross-linking agent. In some embodiments, polyacrylic acids may be used. In some configurations, the cross-linked cellulosic fibers may be crimped, twisted, or curled, or a combination thereof including crimped, twisted, and curled.

In some configurations, one or both of the upper acquisition layer and lower acquisition layer may include a nonwoven, which may be hydrophilic. Further, according to some configurations, one or both of the upper acquisition layer and lower acquisition layer may comprise chemically cross-linked cellulosic fibers, which may or may not form part of a nonwoven material. In some embodiments, the upper acquisition layer may comprise a nonwoven, without the cross-linked cellulosic fibers, and the lower acquisition layer may comprise the chemically cross-linked cellulosic fibers. Further, in some configurations, the lower acquisition layer may comprise the chemically cross-linked cellulosic fibers mixed with other fibers such as natural or synthetic polymeric fibers. According to some embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof.

Exemplary absorbent assemblies 140, absorbent cores 142, and associated components that may be adapted for use with the present disclosure are described in U.S. Patent Nos. 4,610,678; 4,673,402; 4,888,231; 4,834,735; 4,888,231; 5,260,345; 5,387,207; 5,397,316; 8,603,277; and 8,568,566; and U.S. Patent Publication Nos. 2012/0316046 A1 and 2014/0163504 A1, all of which are hereby incorporated by reference herein.

Taped diapers may be manufactured and provided to consumers in a configuration wherein the front waist region and the back waist region are not fastened, pre-fastened, or connected to each other as packaged, prior to being applied to the wearer. For example, the taped diaper 100T may be folded about a lateral centerline with the interior surface 132 of the first waist region 116 in surface to surface contact with the interior surface 132 of the second waist region 118 without fastening or joining the waist regions together. The rear side panels 104 and 106 and/or the front side panels 108 and 110 may also be folded laterally inward toward the inner surfaces 132 of the waist regions 116 and 118.

With continued reference to FIG. 1B, the absorbent article 100 may also include leg gasketing elements 156. It is to be appreciated that the leg gasketing elements 156 can be and are sometimes also referred to as leg cuffs, leg bands, side flaps, barrier cuffs, elastic cuffs or gasketing cuffs. The leg gasketing elements 156 may be elasticized and may be configured in various ways to help reduce the leakage of body exudates in the leg regions. Example leg gasketing elements 156 may include those described in U.S. Patent Nos. 3,860,003; 4,909,803; 4,695,278; 4,795,454; 4,704,115; and U.S. Patent Publication No. 2009/0312730 A1, which are all incorporated by reference herein.

As shown in FIG. 1B, the absorbent article 100 may include longitudinally extending and laterally opposing leg gasketing elements 156 that are disposed on the interior surface 132 of the chassis 102 that faces inwardly toward the wearer and contacts the wearer. Each leg gasketing element 156 may have a first side edge 157 and a second side edge 159, wherein the first side edge 157 is positioned laterally inboard of the second side edge 159. The leg gasketing elements 156 may also overlap the absorbent assembly 140, wherein the first side edges 157 extend laterally inward of the respective side edges 152, 154 of the absorbent assembly 140. In some configurations, the leg gasketing elements 156 may not overlap the absorbent assembly 140. It is to be appreciated that the leg gasketing elements 156 may be formed in various ways, such as for example, by folding portions of the chassis 102 laterally inward, i.e., toward the longitudinal axis 124, to form both the respective leg gasketing elements and the side edges 128 and 130 of the chassis 102. In another example, the leg gasketing elements 156 may be formed by attaching an additional layer or layers to the chassis 102 at or adjacent to each of the respective side edges and of the chassis. Each of the leg gasketing elements 156 may be joined to the interior surface 132 of the chassis and/or the absorbent assembly 140 in leg gasketing element attachment zones in the front waist region 116 and in leg gasketing element attachment zones in the back waist region 118. The leg gasketing elements 156 may extend to the same longitudinal extent as the absorbent article 100 or alternatively the leg gasketing elements 156 may have a longitudinal extent that is less than the absorbent article 100. In some configurations, the leg gasketing elements may be configured to define inner cuffs, outer cuffs, or both inner and outer cuffs.

The absorbent article 100 may also include one or more waist panels 158, such as shown in FIGS. 1A and 1B. The waist panel 158 may provide improved fit and containment and may define a portion or zone of the absorbent article 100 that may elastically expand and contract to dynamically fit a wearer's waist. In some configurations, the absorbent article 100 may include a single waist panel 158, which may be positioned in the second waist region 118. In some configurations, the absorbent article 100 may include more than one waist panel 158. For example, the absorbent article may comprise a first waist panel positioned in the second waist region 118 and a second waist panel 158 positioned in the first waist region 116. The waist panel 158 may be constructed in a number of different configurations including those described in U.S. Patent Nos. 4,515,595 and 5,151,092; and U.S. Patent Publication Nos. 2020/0375807 A1; 2020/0375815 A1; 2021/0128366 A1; 2021/0128369 A1; and 2024/0148562 A1, which are all incorporated by reference herein. It is also to be appreciated that in some configurations, the waist panel 158 may be bonded with the wearer facing surface 132 of the topsheet 138, such as shown in FIGS. 1A and 1B. In some configurations, such as shown in FIGS. 1C and 1D, the waist panel 158 may be bonded with the garment facing surface 134 of the backsheet 136. In particular, FIG. 1C shows a plan view of an absorbent article 100 with the portion of the diaper that faces away from a wearer oriented towards the viewer, and FIG. 1D shows a plan view of the diaper 100 with the portion of the diaper that faces toward a wearer oriented towards the viewer.

It is to be appreciated that the waist panels 158 herein may be configured in various ways and may include one or more elastic materials, such as for example, elastic film and/or elastic strands. In some configurations, the waist panel 158 may be configured as a laminate of two more substrates. For example, the waist panel 158 may be configured as an elastic film and/or elastic strands bonded in between two or more nonwoven substrates and/or may be bonded with one or more nonwoven substrates. For example, the waist panel 158 may be configured as a bi-laminate with an elastic film bonded and/or elastic strands bonded with a single nonwoven substrate. In another example, the waist panel 158 may be configured as an elastic film and/or elastic strands bonded between two or more substrates, wherein the substrates may comprise nonwovens. It is also to be appreciated that nonwoven substrates of the waist panel 158 may be of the same or different material and/or basis weights and may be configured as an elastomeric nonwoven or a non-elastic nonwoven. In some configurations, one more nonwoven substrates of the waist panel 158 may be of the same or different material and/or basis weights as one more nonwoven substrates of the topsheet 138, backsheet 136, and/or leg gasketing elements 156.

It is to be appreciated that components of the waist panel 158 may be bonded together in various ways, such as for example, by adhesive bonds, ultrasonic bonds, pressure bonds, thermal bonds, or combinations thereof. It is to be appreciated that components of the waist panel 158 may be bonded together with adhesive applied in various ways, such as for example, as a spray nozzle and/or a slot coating device. In some configurations, components of the waist panel 158 may be continuously bonded with adhesive or bonded discontinuously with a patterned adhesive. In some configurations, the adhesive may be applied in accordance with the apparatuses and/or methods disclosed in U.S. Patent Nos. 8,186,296; 9,265,672; 9,248,054; and 9,295,590 and U.S. Patent Publication No. 2014/0148773 A1, which are all incorporated by reference herein. In some configurations, components of the waist panel 158 may be mechanically (pressure) bonded with the application of pressure (and optionally heat) in various ways, such as for example, the mechanical bonding devices and methods disclosed in in U.S. Patent Nos. 4,854,984; 6,248,195; 8,778,127; 9,005,392; 9,962,297; and 10,052,237, which are all incorporated by reference herein. In some configurations, components of the waist panel 158 may be mechanically (pressure) bonded with the use of ultrasonic bonding methods configured in various ways, such as for example linear or rotary type configurations, and such as disclosed for example in U.S. Patent Nos. 3,113,225; 3,562,041; 3,733,238; 5,110,403; 6,036,796; 6,508,641; and 6,645,330, which are all incorporated by referenced herein. It is to be appreciated that the waist panel 158 may be formed with various types of bond configurations, such as disclosed, for example, in U.S. Patent Nos. 6,572,595; 6,830,800; 7,087,287; and 7,803,244; and U.S. Patent Publication Nos. 2018/0042778 A1; 2018/0042787 A1; 2018/0042779 A1; and 2018/0042780 A1, which are all incorporated by reference herein.

It is to be appreciated that the waist panel 158 may be located in various positions relative to the wearer facing surfaces 132 and garment facing surfaces 134 of various absorbent article components. For example, as shown in FIGS. 1A and 1B, the waist panel 158 may be positioned on and bonded with the wearer facing surface 132 of the topsheet 138. As further described below, the waist panel 158 may be bonded with the topsheet 138 so as to form a pocket 192 between the waist panel 158 and the topsheet 138. In addition, the pocket 192 may comprise an opening defined by an edge of the waist panel that is not bonded with the topsheet 138. It also to be appreciated that configurations with a first waist panel positioned in the second waist region 118 and a second waist panel 158 positioned in the first waist region 116, one or both of first and second waist panels may be bonded with the chassis 102 so as to form a pocket 192.

It is further to be appreciated that the waist panels 158 herein may comprise elastic materials, such as elastic strands and/or films, configured to form varying stretch profiles of the waist panel 158. For example, elastic materials may be configured to define relatively high and low zones of contraction forces in the waist panel 158, such as disclosed in U.S. Patent Nos. 10,398,608 and 10,406,040, which are both incorporated by reference herein. In some configurations, the waist panel 158 may comprise one of more zones of relatively high contraction forces positioned longitudinally inboard of one or more zones of relatively low contraction forces. In some configurations, the waist panel 158 may be positioned on and bonded with the garment facing surface 134 of the backsheet 136, as shown in FIGS. 1C and 1D. In some configurations, the waist panel 158 may be positioned entirely longitudinally outboard of the absorbent assembly 140. And in some configurations, a portion of the waist panel 158 may overlap a portion of the absorbent assembly 140.

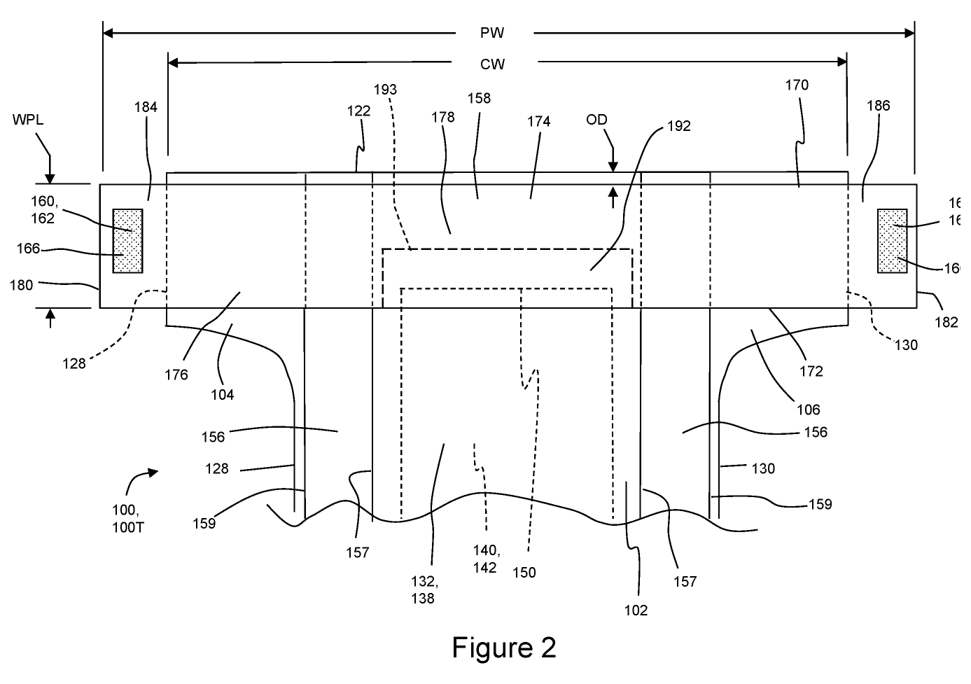

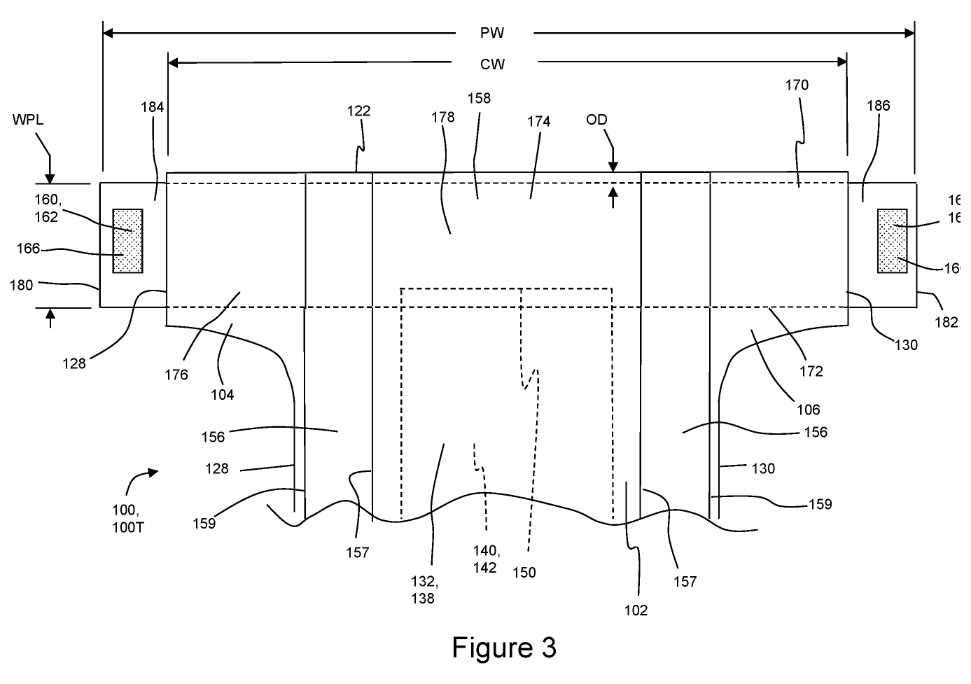

As shown in FIGS. 2 and 3, the waist panels 158 herein may comprise a first lateral edge 170 and a second lateral edge 172, wherein the second lateral edge 172 is positioned longitudinally inward relative the first lateral edge 170. As such, the first lateral edge 170 may be configured as an outboard lateral edge, and the second lateral edge 172 may be configured as an inboard lateral edge. In addition, the waist panel 158 may comprise a first longitudinal end region 174 adjacent the first lateral edge 170 and a second longitudinal end region 176 adjacent the second lateral edge 172, wherein the first and second longitudinal end regions 174, 176 are separated by a central region 178. The first and second lateral edges 170, 172 may be connected with and separated by a first longitudinal edge 180 and a second longitudinal edge 182. As such, the waist panels 158 may also include a first lateral end region 184 adjacent the first longitudinal edge 180 and a second lateral end region 186 adjacent the second longitudinal edge 182, wherein the first and second lateral end regions 184, 186 are separated by the central region 178. In some configurations, the first lateral edge 170, second lateral edge 172, first longitudinal edge 180, and/or second longitudinal edge 182 may be defined by a fold line, wherein one or more layers of waist panel 158 may have been folded onto itself or another layer during assembly. In some configurations, the first lateral edge 170, second lateral edge 172, first longitudinal edge 180, and/or second longitudinal edge 182 may be defined by unfolded edge or a cut line, wherein one or more layers of waist panel 158 may have been cut or trimmed during assembly.

It is to be appreciated that the waist panels 158 herein may be configured with various shapes and/or sizes. For example, as shown in FIGS. 2 and 3, the waist panels 158 may comprise a width PW extending between first and second longitudinal edges 180, 182. The waist panels 158 may comprise a length WPL extending between first and second lateral edges 170, 172. In some configurations, the waist panel 158 may be sized and/or positioned such that the first and second longitudinal edges 180, 182 of the waist panel 158 are located laterally outboard of the first and second side edges 128, 130 of chassis 102. For example, as shown in FIGS. 2 and 3, the chassis 102 may comprise a width CW in the extending between the first side edge 128 and the second side 130 in the waist region where the waist panel 158 is positioned. In turn, the width PW of the waist panel 158 may be larger than the width CW of the chassis 102. In some configurations, the width PW of the waist panel 158 may be from about 10 mm to about 100 mm greater than the width CW of the chassis 102, specifically reciting all 1 mm increments within the above-recited range and all ranges formed therein or thereby.

With continued reference to FIGS. 2 and 3, it is to be appreciated that the waist panels 158 may be located in various lateral and longitudinal positions relative to various absorbent article components. In some configurations, the waist panel 158 may be positioned longitudinally inboard from the second waist edge 122 of the absorbent article 100 and/or toward or overlapping the second lateral edge 150 of the absorbent assembly 140. In some configurations, the first lateral edge 170 of the waist panel 158 may be positioned longitudinally inboard from the second waist edge 122 by an offset distance OD that is greater than zero. In some configurations, the offset distance OD may be at least 5 mm. In some configurations, the first lateral edge 170 of the waist panel 158 may be coterminous with the second waist edge 122 such that the offset distance OD is zero.

It is to be appreciated that the waist panels 158 may be bonded with the chassis 102 in various ways, such as for example, by adhesive bonds, ultrasonic bonds, pressure bonds, thermal bonds or combinations thereof, such as disclosed for example in U.S. Patent Publication Nos. 2020/0375807 A1; 2020/0375815 A1; 2021/0128369 A1; and 2021/0128366 A1, which are incorporated by reference. It is also to be appreciated that the waist panel 158 may be bonded with the chassis 102 with adhesive applied in various ways, such as for example, as a spray nozzle and/or a slot coating device. In some configurations, the waist panels 158 may be continuously bonded with the chassis 102 with adhesive or bonded discontinuously with a patterned adhesive. In some configurations, the adhesive may be applied in accordance with the apparatuses and/or methods disclosed in U.S. Patent Nos. 8,186,296; 9,265,672; 9,248,054; and 9,295,590 and U.S. Patent Publication No. 2014/0148773 A1, which are all incorporated by reference herein. In some configurations, the waist panels 158 may be mechanically (pressure) bonded with the chassis 102 with the application of pressure (and optionally heat) in various ways, such as for example, the mechanical bonding devices and methods disclosed in in U.S. Patent Nos. 4,854,984; 6,248,195; 8,778,127; 9,005,392; 9,962,297; and 10,052,237, which are all incorporated by reference herein. In some configurations, the waist panels 158 may be mechanically (pressure) bonded with the chassis 102 with the use of ultrasonic bonding methods configured in various ways, such as for example linear or rotary type configurations, and such as disclosed for example in U.S. Patent Nos. 3,113,225; 3,562,041; 3,733,238; 5,110,403; 6,036,796; 6,508,641; and 6,645,330, which are all incorporated by reference herein.

As discussed above, the waist panel 158 may be bonded with the topsheet 138 so as to form a pocket 192 between the waist panel 158 and the topsheet 138. As shown in FIG. 2, one or more regions of the waist panel 158 may be bonded with the topsheet 138 and one or more regions of the waist panel 158 may not be bonded (unattached) with the topsheet 136, thereby forming a pocket 192 (generically illustrated by a rectangle with a dashed border 193) between the waist panel 158 and the topsheet 136. As such, a portion of the second longitudinal end region 176 and at least a portion the second lateral edge 172 may be unattached to the topsheet 138 and/or leg gasketing elements 156. It is to be appreciated that the waist panels 158 herein may be configured with one or more pockets 192 with various shapes and/or sizes.

As discussed above, the waist panels 158 herein may be elastic and may comprise at least one direction of stretch. In some configurations, the direction of stretch may be laterally oriented between the first longitudinal edge 180 and the second longitudinal edge 182. As such, the waist panel 158 may provide improved fit and containment and may define a portion or zone of the absorbent article 100 that may elastically expand and contract to dynamically fit a wearer's waist. As shown in FIGS. 1A-1D, the waist panel 158 may be positioned on the chassis 102 to elasticize the second waist region 118. In some configurations, the waist panel 158 may be configured to elasticize the second waist region 118, including all or portions of the side panels 104, 106, extending laterally between the first side edge 128 and the second side edge 130. In some configurations, the waist panel 158 be configured to elasticize an entire lateral chassis width CW, contiguously and without gaps or breaks in elasticity, of the chassis 102 in the second waist region 118, including the side panels 104, 106, extending laterally between the first side edge 128 and the second side edge 130. In some configurations, the waist panel 158 may comprise elastic for the entire width PW or may be elastic along a distance less than the entire width PW. It is also to be appreciated that the waist panel 158 may comprise various types of stretch characteristics. Such stretch characteristics may comprise a percent contraction or a percent elongation. In some configurations, the waist panel 158 may be configured to stretch by about 50 mm to about 150 mm from an uncontracted state to a fully stretched state, specifically reciting all 1 mm increments within the above-recited range and all ranges formed therein or thereby. In some configurations, the stretch characteristics of the waist panel 158 may be the same or may vary between the first lateral edge 170 and the second lateral edge 172 and/or the between the first longitudinal edge 180 and the second longitudinal edge 182.

The absorbent articles 100 may also include various configurations of fasteners to enable fastening of the front waist region 116 and the back waist region 118 together to form a closed waist circumference and leg openings once the absorbent article is positioned on a wearer. For example, as shown in FIGS. 1A-1D, the absorbent article 100 may comprise fasteners 160, such as first and second fasteners 162, 164, positioned on the waist panel 158. The absorbent article 100 may or may not include fasteners on first and second front side panels 108, 110.

With continued reference to FIGS. 1A-1D, the first fastener 162 may be positioned in the first end region 184 on the wearer facing surface 132 of waist panel 158, and the second fastener 164 may positioned in the second end region 186 on the wearer facing surface 132 of waist panel 158. The fasteners 162, 164 may comprise a separate element connected with the waist panel 158 in various ways, such as mechanical bonding, adhesive bonding, or both. In some configurations, the fasteners 162, 164 may be integrally formed from materials of the waist panel 158. For example, hooks may be integrally formed from a waist panel material, which may for example be in the form of a nonwoven, such as disclosed for example in U.S. Patent Nos. 6,478,784; 8,784,722; 11,376,170; and 11,576,822; and U.S. Patent Publication No. 2018/0141266 A1, which are all incorporated herein by reference. In some configurations, the waist panel 158 may be configured with relatively stiff first and second end regions 184, 186 adjacent the first and second fasteners 162, 164, respectively. Such stiffened regions may be formed with the materials of the first and second fasteners 162, 164 and/or with additional substrates bonded with together in the waist panel 158.

The fasteners 162, 164 may form a portion of or may be permanently bonded, adhered or otherwise joined directly or indirectly to the waist panel 158 and may be positioned laterally inward from the side edges 180, 182. Depending on the relative size and/or shape of the first and second rear side panels 104, 106 and the waist panel 158, the fasteners 162, 164 may be positioned laterally outboard of the first and second side edges 128, 130. In some configurations, the fasteners 162, 164 may be positioned laterally inboard of or overlapping with the first and second side edges 128, 130. The fasteners 162, 164 may also be permanently bonded or joined with the waist panel 158 in various ways, such as for example, by adhesive bonds, sonic bonds, pressure bonds, thermal bonds or combinations thereof, such as disclosed for example, U.S. Patent No. 5,702,551, which is incorporated by reference herein.

It is to be appreciated that fasteners 160, such as the first fastener 162 and/or the second fastener 164, may include various types of releasably engageable fasteners. The first and second fasteners 162 and/or 164 may also include various types of refastenable fastening structures. For example, the first and second fasteners 162 and 164 may include mechanical fasteners, 166, in the form of hook fasteners, loop fasteners, macrofasteners, buttons, snaps, tab and slot fasteners, tape fasteners, adhesive fasteners, cohesive fasteners, magnetic fasteners, hermaphroditic fasteners, and the like. Some examples of fastening systems and/or fasteners 162, 164 are discussed in U.S. Patent Nos. 3,848,594; 4,662,875; 4,846,815; 4,894,060; 4,946,527; 5,151,092; 5,221,274; 5,242,436; 6,251,097; 6,669,618; 6,432,098; and U.S. Patent Publication Nos. 2007/0078427 A1; 2007/0093769 A1; and 2021/0145661 A1, which are all incorporated by reference herein.

As previously mentioned, the fasteners 162 and 164 may also be adapted to releasably and/or refastenably engage or connect with another portion of the absorbent article 100. For example, as shown in FIGS. 1A-1D, the absorbent article 100 may include a connection zone 168, sometimes referred to as a landing zone, in the first waist region 116. As such, when the taped absorbent article 100 is placed on a wearer, the fasteners 162 and 164 may be pulled around the waist of the wearer and connected with the connection zone 168 in the first waist region 116 to form a closed waist circumference and a pair of laterally opposing leg openings. It is to be appreciated that the connection zone 168 may be constructed from a separate substrate that is connected with the chassis 102 of the absorbent article. In some embodiments, the connection zone may be integrally formed as part of the backsheet 136 of the absorbent article 100, such as described in U.S. Pat. Nos. 5,735,840 and 5,928,212, which are both incorporated by reference herein.

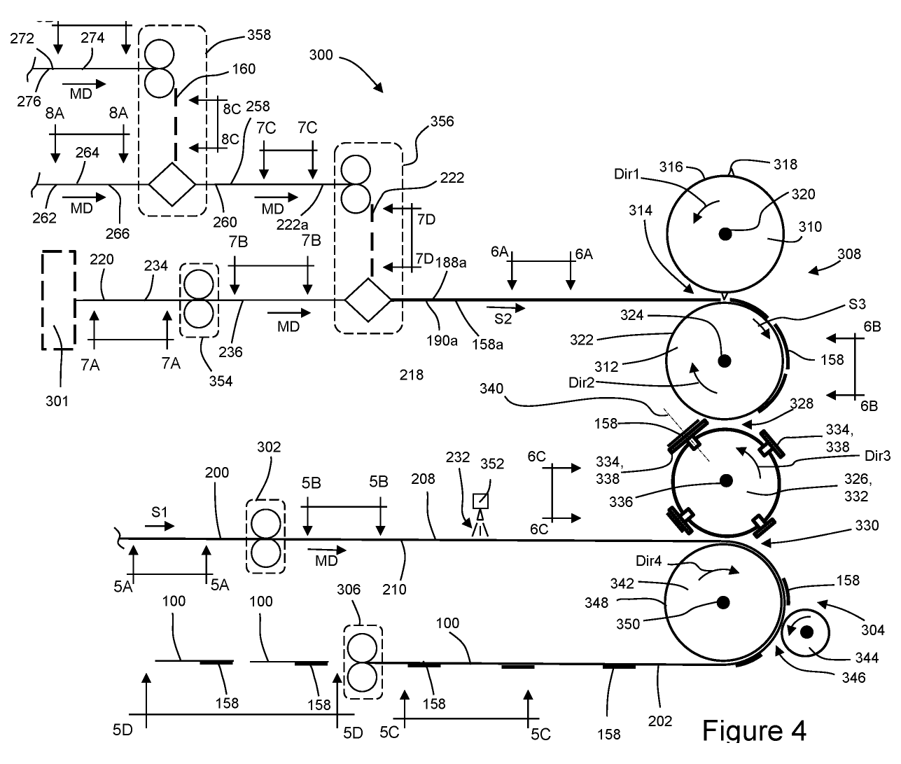

It is to be appreciated that absorbent articles 100 may be assembled with various components, including waist panels 158, described herein in various ways. Thus, in the context of the previous discussion, the apparatuses and methods herein may be adapted to bond discrete waist panels 158 under tension to an advancing primary carrier substrate 200 during the assembly of an absorbent article 100 having integrally formed side panels in a waist region, such as the first and second rear side panels 104, 106 described above. As such, waist panels 158 may be bonded in a stretched state with topsheets 138 or backsheets 136 to elasticize the side panels 104, 106 as well as entire the waist region 118 located laterally between the side panels 104, 106. As discussed in more detail below with reference to FIG. 4, the apparatuses and methods herein may be utilized to assemble and bond waist panels 158 to primary carrier substrates 200 that may be configured as topsheets 138 or backsheets 136 during the manufacture of absorbent articles 100. When bonded with a topsheet 138, the waist panel 158 may also be configured to form a pocket 192 between the waist panel 158 and the topsheet 138. It is to be appreciated that the systems and methods disclosed herein are applicable to work with various types of converting processes and/or machines.

FIG. 4 shows a schematic representation of a converting process including an apparatus or system 300 that may assemble absorbent articles 100Twith integrally formed side panels 104, 106 and a waist panel 158 that elasticizes a central portion of the waist region 118 as well as the integrally formed side panels 104, 106. As discussed below, the system 300 bonds discrete waist panels 158 with an advancing primary carrier substrate 200 to form a laminate 202. Some aspects and variations of the apparatuses 300 and associated assemblies relating shown in FIG. 4 relating to the waist panel 158 are disclosed in U.S. Patent Publication Nos. 2020/0375807 A1; 2020/0375815 A1; 2021/0128366 A1; 2021/0128369 A1; and 2024/0148562 A1, which are all incorporated by reference herein.

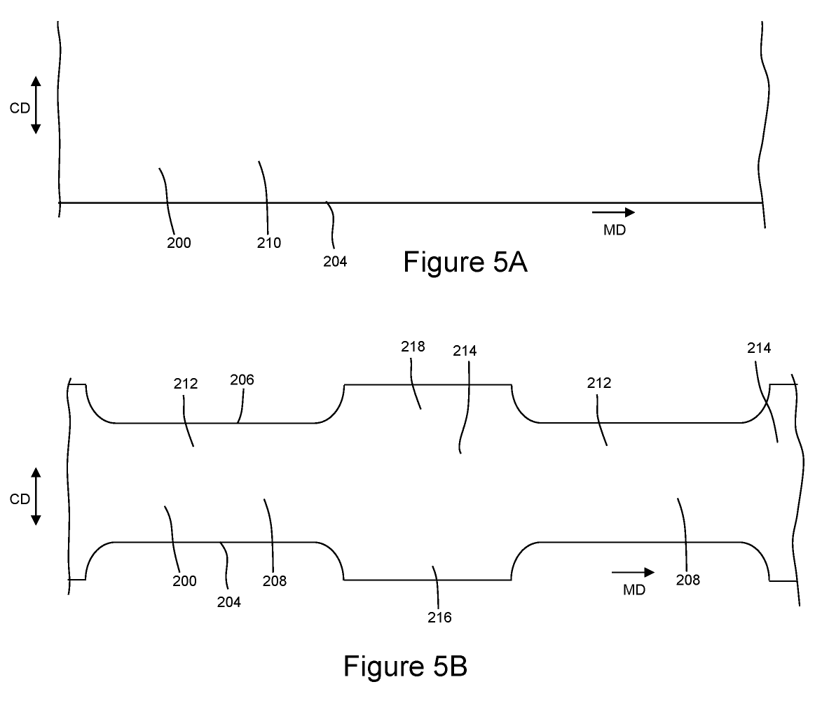

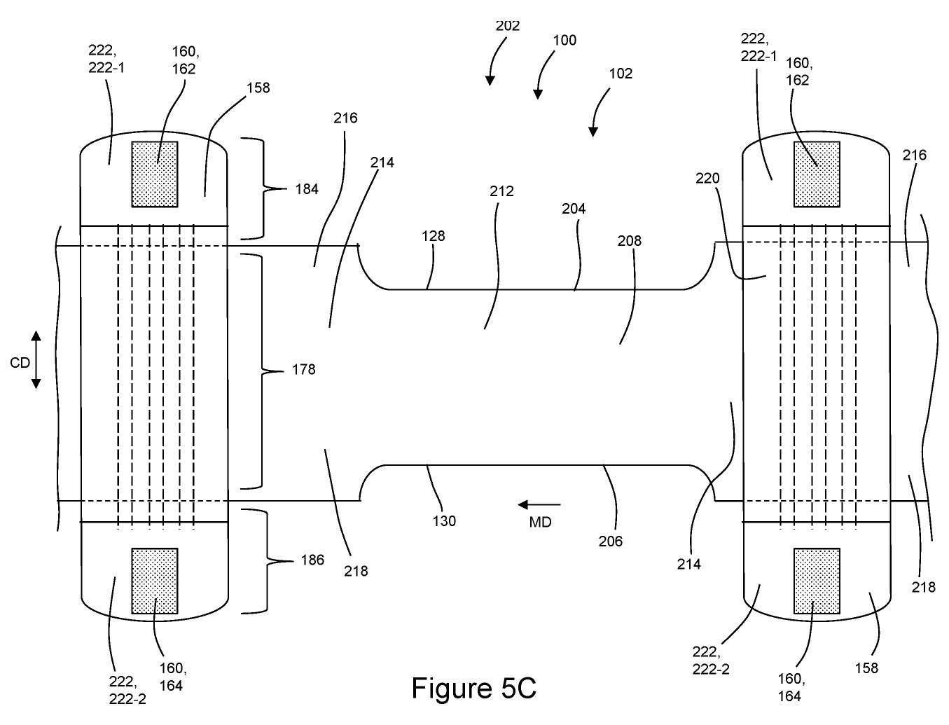

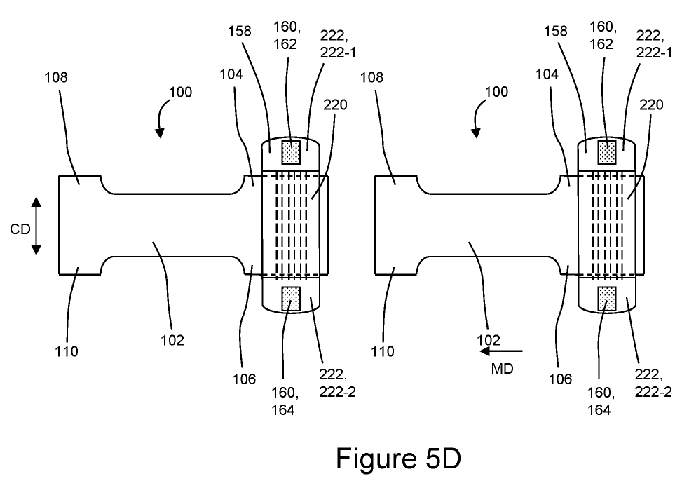

As shown in FIGS. 4 and 5A, the primary carrier substrate 200 may advance in a machine direction MD at a first speed S1. The primary carrier substrate 200 comprises a first longitudinal edge 204 and a second longitudinal edge 206 separated from the first longitudinal edge 206 in a cross direction CD to define a width. The primary carrier substrate 200 also includes a first surface 208 and an opposing second surface 210. In some configurations, the primary carrier substrate 200 may advance to an edge trimming station 302 adapted to intermittently trim material from the primary carrier substrate 200 to contour the first edge 204 and the second edge 206. It is to be appreciated that the edge trimming station 302 may be configured in various ways, such as, for example, a rotating knife and anvil roll. As shown in FIG. 5B, the primary carrier substrate 200 advances from the edge trimming station 302, and the contoured first and second edges 204, 206 of the primary carrier substrate 200 define relatively narrow regions 212 of the primary carrier substrate 200 arranged intermittently between relatively wide regions 214 of the primary carrier substrate 200 along the machine direction MD, wherein the relatively wide regions 214 define integrally formed first and second wing panels 216, 218.

As shown in FIGS. 4, 5B, and 5C, the primary carrier substate 200 may advance to a bonding station 304 where discrete waist panels 158 are bonded under tension with the first surface 208 of the primary carrier substrate 200 to form a laminate 202. As shown in FIG. 5C, each discrete waist panel 158 may comprise a first surface 188 and an opposing second surface 190. As discussed above, the waist panel 158 also includes a first end region 184, a second end region 186, and a central region 178 separating the first end region 184 from the second end region 186. In addition, each waist panel 158 may comprise a first fastener 162 on the first surface 188 of the first end region 184 and a second fastener 164 on the first surface 188 of the second end region 186. The waist panels 158 may be positioned to extend in the cross direction CD along the relatively wide regions 214 of the primary carrier substrate 200. For example, with continued reference to FIG. 5C, each waist panel 158 may extend with a direction of stretch oriented along the a cross direction CD such that the first end region 184 extends outboard from the first edge 204 of the first wing panel 216, and the second end region 186 extends outboard from the second edge 206 of the second wing panel 218 of the primary carrier substrate 200.

In the context of components of absorbent articles 100 discussed above and assembly processes thereof, the primary carrier substrate 200 may be configured as a continuous topsheet 138, a continuous backsheet 136, or a continuous laminate of a combined topsheet 138 and backsheet 136. As such, the first surface 208 of the primary carrier substrate 200 may correspond with the wearer facing surface 132 of the topsheet or the garment facing surface 134 of the backsheet 136. In some configurations, the waist panel 158 may be bonded with the garment facing surface 134 of the backsheet 136, wherein the wearer facing surface 132 of the backsheet 136 may have been previously bonded with a topsheet 138 or may be subsequently bonded with a topsheet 138. In another example, the waist panel 158 may be bonded with the wearer facing surface 132 of the topsheet 136, wherein the garment facing surface 134 of the topsheet 138 may have been previously bonded with a backsheet 136 or may be subsequently bonded with a backsheet 136.

In some configurations, the primary carrier substrate 200 may also include leg cuffs 156 positioned adjacent the first longitudinal edge 204 and the second longitudinal edge 206. As such, portions of the waist panels 158 may also be bonded with the leg cuffs 156. In some configurations, the waist panels 158 may be bonded with the primary carrier substrate 200 and leg cuffs 156 may subsequently be bonded with the primary carrier substrate 200. The leg cuffs 156 may be positioned relative to the waist panel 158 such that the leg cuffs 156 may or may not partially cover or overlap opposing end portions of the waist panel 158. In some configurations, the leg cuffs 156 may be sandwiched between the waist panel 158 and the primary carrier substrate 200. And in some configurations, the waist panel 158 may be sandwiched between the leg cuffs 156 and the primary carrier substrate 200.

With continued reference to FIG. 4, after the discrete waist panel 158 is bonded with the primary carrier substrate 200 to create the laminate 202, the laminate 202 may continue to advance in the machine direction MD from the bonding station 304 and may be subjected to additional converting operations, such as cutting, folding, and/or packaging operations. In some configurations, the laminate 202 may define a continuous length of absorbent articles 100 or chassis 102 or may be combined with additional substrates and/or components to define a continuous length of absorbent articles. For example, as shown in FIGS. 4, 5C, and 5D, the continuous length of absorbent articles 100 may advance to a final cutting station 306 to be subjected to a final knife cut that separates discrete absorbent articles 100 from the continuous length of absorbent articles 100. In some configurations, the final knife cut operation may apply a cut line the extends through the first and second wing panels 216, 218 of the primary carrier substate 200 adjacent the waist panel 158. In turn, the wing panels 216, 218 may be divided by the final knife cut line to define front side panels 108, 110 and rear side panels 104, 106 on each discrete absorbent article 100, such as shown in FIG. 5D. It is also to be appreciated that such final knife cut operations may be configured to apply straight and/or curved cut lines through the primary carrier substrate 200.

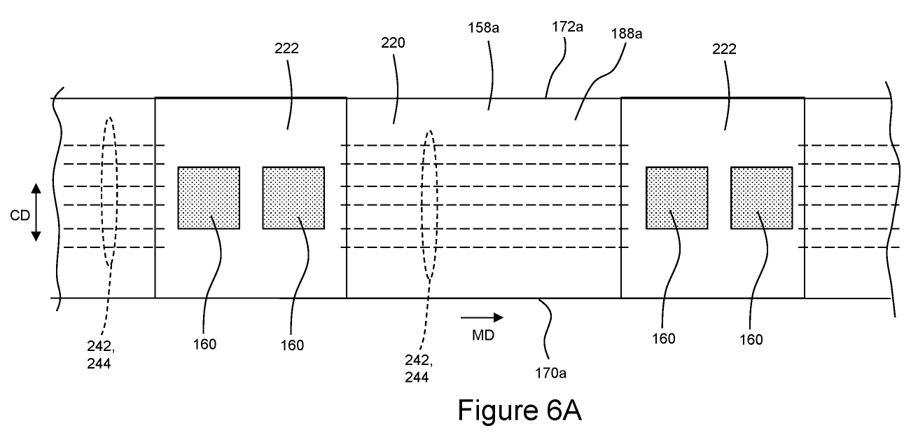

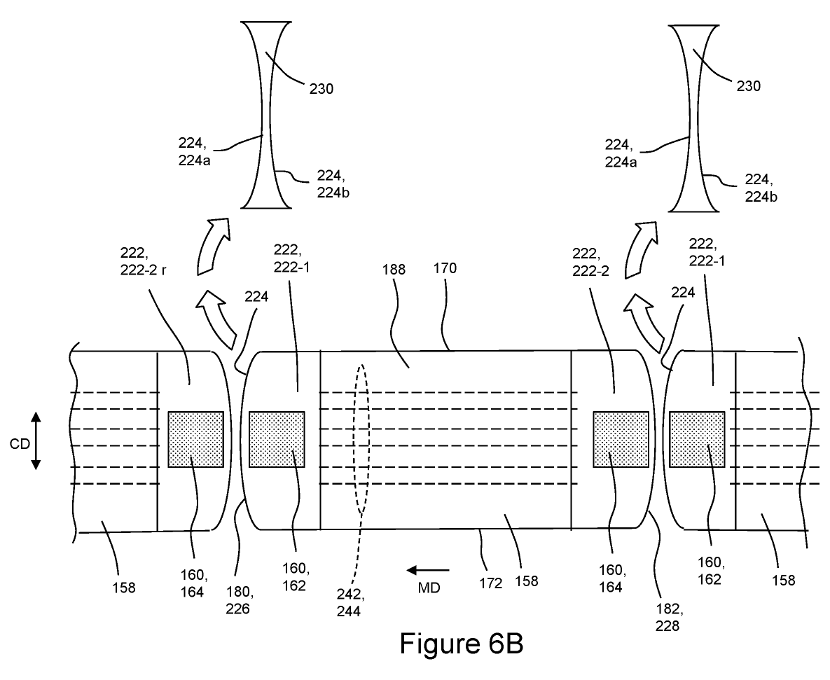

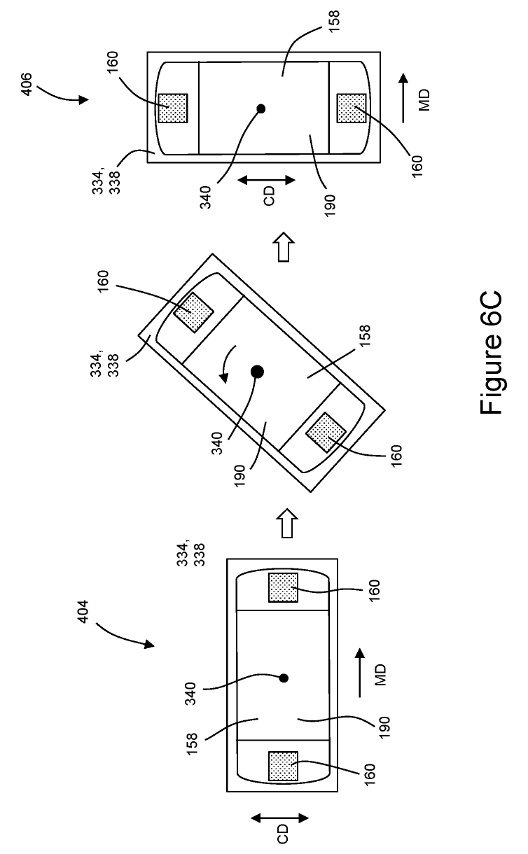

It is to be appreciated that the method for providing and bonding discrete waist panels 158 to the primary carrier substrate 200 may be configured in various ways. For example, as shown in FIGS. 4 and 6A, a continuous waist panel laminate 158a may be advanced at a second speed S2 in the machine direction MD. The continuous waist panel laminate 158a is stretchable in at least one direction and is oriented such that the continuous waist panel laminate 158a is stretchable in the machine direction MD. In some configurations, the second speed S2 may less than the first speed S1 of the primary carrier substrate 200. As shown in FIG. 6A, the continuous waist panel laminate 158a may comprise an elastic laminate 220 with fastener panels 222 intermittently spaced from each other in the machine direction MD on the elastic laminate. At least one fastener 160 may be positioned on each fastener panel 222. The waist panel laminate 158a also comprises a first longitudinal edge 170a and a second longitudinal edge 172a separated from the first longitudinal edge 170a in the cross direction CD to define a width. The continuous waist panel laminate 158a also includes a first surface 188a and an opposing second surface 190a.

With reference to FIGS. 4, 6A, and 6B, the continuous waist panel laminate 158a may advance in the machine direction MD to a cutting device 308 that separates the waist panel laminate 158a along at least one line of separation 224 extending through the elastic laminate 220 and the fastener panels 222 to form discrete waist panels 158. As such, the discrete waist panels 158 each include a first edge 180 and a second edge 182 and defines a length in the machine direction MD extending between the first edge 180 and the second edge 182. The waist panel 158 also includes first and second longitudinal edges 170, 172 that correspond with the longitudinal edges 170a, 172a of the continuous waist panel laminate 158a. In addition, the waist panel 158 includes first and second surfaces 188, 190 that correspond with the first and second surfaces 188a, 190a of the continuous waist panel laminate 158a. As shown in FIG. 6B, the discrete waist panel 158 is also formed with the first end region 184 adjacent the first edge 180 and the second end region 186 adjacent the second edge 182. When positioned on the cutting device 304, the waist panel 158 may be oriented such that the first edge 180 is a leading edge 226 and the second edge 182 is a trailing edge 228, and the second end region 186 may be separated from the first end region 184 in the machine direction MD by the central region 174. It is to be appreciated that the continuous waist panel laminate 158a and discrete waist panels 158 herein may be configured in various ways and may include one or more elastic materials, such as for example, elastic film and/or strands.

With continued reference to FIGS. 6A and 6B, each waist panel 158 may comprise a first portion 222-1 of a first separated fastener panel 222 on the first surface 188 of the first end region 184 and a second portion 222-2 of a second separated fastener panel 222 on the first surface 188 of the second end region 186. As shown in in FIG. 6A, the fastener panels 222 of the waist band laminate 158a may include first and second fasteners 162, 164 separated from each other along the machine direction MD. As such, the cutting device 308 may operate to cut through the fastener panels 222 between the first fastener 162 and the second fastener 164. In some configurations, the cutting device 308 may cut through a fastener 160 to form a first fastener 162 and a second fastener 164.

It is to be appreciated that the cutting device 308 may be configured in various ways to separate the discrete waist panels 158 from the continuous waist panel laminate 158a. For example, as shown in FIG. 4, the cutting device 308 may include a knife roll 310 positioned adjacent an anvil roll 312 to define a nip 314 therebetween. The knife roll 310 may include an outer circumferential surface 316 and one or more blades 318 adapted to rotate about an axis 320 in a first direction Dir1. The anvil roll 312 may include an outer circumferential surface 322 adapted to rotate about an axis 324 in a second direction Dir2 opposite the first direction Dir1 such that the outer circumferential surface 322 advances at a third speed S3. In some configurations, the third speed S3 is greater than the second speed S2, and as such, the continuous waist panel laminate 158a is stretched in the machine direction MD. With continued reference to FIG. 4, as the continuous waist panel laminate 158a advances through the nip 314 between the knife roll 310 and the anvil roll 312, the blade 318 operates to cut the discrete waist panel 158 from the continuous waist panel laminate 158a. Because the outer circumferential surface 322 of the anvil roll 312 may advance at the third speed S3 that is greater than the second speed S2, the cut discrete waist panel 158 is in a stretched state in the machine direction MD on the outer circumferential surface 322 of the anvil roll 312. The anvil roll 312 may also be configured to apply vacuum pressure to the discrete waist panels 158 to help hold the discrete waist panels 158 on the outer circumferential surface 322 in a stretched state as the anvil roll 312 rotates. It is also to be appreciated that one or more components of the cutting device 308 may be configured to operate at constant and/or variable speeds. For example, the knife roll 310 and/or the anvil roll 312 may be connected with various types of motors, such as servo motors for example, that may rotate the knife roll 310 and/or the anvil roll 312 at constant and/or variable angular velocities.

In some configurations, the third speed S3 may be equal to the first speed S1 of the advancing primary carrier substrate 200. In some configurations, the third speed S3 may be less than or greater than the first speed S1 of the advancing primary carrier substrate 200, and as such, the discrete waist panel 158 may be accelerated or decelerated downstream of the anvil roll 312 from the third speed S3 to the first speed S1 before being combined with the primary carrier substrate 200. When the first speed S1 of the primary carrier substrate 200 is greater than the third speed S3, the discrete waist panels 158 are accelerated from the third speed S3 to the first speed S1 before bonding with the primary carrier substrate 200. By accelerating discrete waist panels 158 from the third speed S3 to the first speed S1, trailing edges 226 (or leading edges 228) of consecutively cut discrete waist panels 158 may be separated from each other in the machine direction MD.

In some configurations, the blade 318 may be configured such that resulting cut lines and corresponding leading edges 226 and trailing edges 228 of the discrete waist panels 158 may be straight and/or curved. In some configurations, such as illustrate in FIGS. 4 and 6B, the knife roll 310 may be configured to perform a die cut on the continuous waist panel laminate 158a wherein trim pieces 230 are cut from the waist panel laminate 158a and wherein the each trim piece extends between a first line of separation 224a and a second line of separation 224b. The cutting device 308 may also be adapted to cut the discrete waist panels 158 such that material along the cut line adjacent leading edges 226 and trailing edges 228 is fused and/or pressure bonded together. It is also to be appreciated that the positions of the knife roll 310 and anvil roll 312 may be opposite to that which is illustrated in FIG. 4, and as such, the discrete waist panels 158 may remain on the outer circumferential surface 316 of the knife roll 310 as opposed to the anvil roll 312.

It is also to be appreciated that the cutting device 308 may be configured to convey and/or cut the discrete waist panels 158 in different ways. In other configurations, the cutting device 308 may be adapted to create a line of separation 224 in the form of a line of weakness comprising perforations in the waist panel laminate 158a. In turn, the cutting device 308 may also be adapted to separate discrete waist panels 158 from the waist panel laminate 158a by tearing the waist panel laminate 158a along the lines of weakness, such a disclosed for example in U.S. Patent No. 6,599,384, which is incorporated herein by reference. It is also to be appreciated that lines of weakness may be configured with tensile strengths so as to mitigate unintentional separation during manufacturing and web handling operations, such as disclosed for example in U.S. Patent Application No. 18/967,768, which is incorporated by reference.

With continued reference to FIG. 4, the apparatus 300 may include a rotatable transfer device 326 that transfers the discrete waist panels 158 from the cutting device 308 to the bonding station 304, which in turn, combines the waist panels 158 with the primary carrier substrate 200. The transfer device 326 may be configured as turn and repitch device. As such, the transfer device 326 may be configured to turn, rotate, and repitch the discrete waist panels 158. The transfer device 326 may also be configured to stretch the discrete waist panels 158 in the machine direction MD.

As shown in FIG. 4, the transfer device 326 may be configured to transport the stretched discrete waist panels 158 in a machine direction MD from a nip 328 between the anvil roll 312 and the transfer device 326 to a nip 330 between the transfer device 326 and the bonding station 304. However, the transfer device 326 may also be configured to reorient the discrete waist panels 158 from a first orientation 404 on the anvil roll 312 having the direction of stretch in the machine direction MD to a second orientation 406 having the direction of stretch in the cross direction CD. For example, the transfer device 326 may comprise a turn and repitch apparatus 332 that may comprise and one or more transfer members 334 adapted to orbit as indicated by directional arrow Dir3 in a machine direction MD about a first axis 336. In turn, each transfer member 334 may include a carrier surface 338 adapted to engage the discrete waist panels 158. As shown in FIGS. 4 and 6C, the carrier surface 338 may also be rotatable about a second axis 340 as indicated by directional arrow Dir6 between the first orientation 404 and the second orientation 406. The carrier surface 338 may be configured to be in communication with a vacuum source to help hold the waist panels 158 on the carrier surfaces 338 in a stretched state. As such, the carrier surface 338 and the waist panel 158 positioned on the carrier surface 338 orbit about the first axis 326 from the nip 328 to the nip 330. In addition, while orbiting from the nip 328 to the nip 330, the carrier surface 338 and the discrete waist panel 158 are rotated about the second axis of rotation 340 to place the carrier surface 338 and the waist panel 158 in a second orientation. In particular, in the second orientation, the direction of stretch of the waist panel 158 is oriented in the cross direction and is perpendicular or substantially perpendicular to the machine direction MD. For example, FIG. 6C illustrates a waist panel 158 on a carrier surface 338 beginning in the first orientation 404 and being rotated about the second axis 340 to place the waist panel 158 in the second orientation 406. Once the waist panels 158 are transferred to the primary carrier substrate 200 at the nip 330, the bonding station 304 of FIG. 4 may operate to bond the waist panels 158 and the primary carrier substrate 200 together.

It is to be appreciated that the transfer device 326 shown in FIG. 4 may be configured in various ways, for example, such as disclosed in U.S. Patent Application No. 18/492,918; U.S. Patent Nos. 7,587,966; 9,168,182; and 9,737,442; and U.S. Patent Publication Nos. 2021/0267812 and 2022/0008256; which are all incorporated by reference herein. It is also to be appreciated that the transfer device 326 may be configured as a combination of a turning device that performs a step of reorienting the wait panels 158 and a separate device that performs a repitching step. The transfer device may also be configured with movable heads configured to adjust the stretch of the waist panels 158, such as disclosed for example in U.S. Patent No. 10,806,635, which is incorporated by reference.

With continued reference to FIG. 4, the primary carrier substrate 200 may advance through the nip 330 between the rotatable transfer device 326 and the bonding station 304. At the nip 330, the discrete waist panels 158 are received from the transfer device 326 onto the first surface 208 of the primary carrier substrate 200 with waist panels 158 stretched in the cross direction CD. In turn, the bonding station 304 bonds the discrete waist panels 158 in the stretched state with the advancing primary carrier substrate 200.

It is to be appreciated that the waist panels 158 may be bonded with the primary carrier substrate 200 in various ways. For example, in some configurations, the waist panels 158 may be bonded with the primary carrier substrate 200 with adhesive. In some configurations, the waist panels 158 may be bonded with the primary carrier substrate 200 with mechanical bonds. And in some configurations, the waist panels 158 may be bonded with the primary carrier substrate 200 with a combination of adhesive and mechanical bonds. When the primary carrier substrate 200 is configured as a topsheet 138, the waist panel 158 may be bonded with the primary carrier substrate 200 so as to form a pocket 192 between the waist panel 158 and the primary carrier substrate 200.

It is also to be appreciated that the bonding station 304 may be configured in various ways. For example, as shown in FIG. 4, the bonding station 304 may be configured with a first roll 342 and counterrotating second roll 344 adjacent the first roll 340 to define a nip 346 therebetween. The first roll 342 includes an outer circumferential surface 348 and rotates about an axis of rotation 350, wherein the first roll 340 may rotate in a fourth direction Dir4 that is opposite the third direction Dir3. In addition, first roll 340 may rotate such that the outer circumferential surface 348 advances at or about the first speed S1. During operation, the primary carrier substrate 200 advances to the nip 330 and onto the outer circumferential surface 348 of the first roll 342. Discrete waist panels 158 in a stretched state are transferred from transfer device 326 to the first surface 208 of the primary carrier substrate 200 at the nip 330. As such, the combined primary carrier substrate 200 and the waist panels 158 may advance through the nip 346, which may be configured to press the primary carrier substate 200 and waist panels 158 together to further secure adhesive bonds between the primary carrier substate 200 and waist panels 158 and/or to mechanically bond the primary carrier substate 200 and waist panels 158 together.

The system 300 may include one or more adhesive applicator devices 352 in various locations of the assembly process to deposit adhesive 232 onto components to bond the primary carrier substrate 200 and the waist panels 158 together. As shown for example in FIG. 4, the system 300 may include an adhesive applicator device 352 that deposits adhesive 232 onto the first surface 208 of the primary carrier substrate 200 before being combined with the waist panels 158 at the nip 330. As such, the adhesive 232 may bond the first surface 208 of the primary carrier substrate 200 with the waist panels 158. In some configurations, the system 300 may include an adhesive applicator 352 adapted to apply adhesive 232 to the second surfaces 190 of waist panels 158 on the transfer apparatus 326. In some configurations, the system may include an adhesive applicator 352 adapted to apply adhesive 232 to the second surface 190a of the waist panel laminate 158a upstream of the nip 314. It is to be appreciated that adhesive applicator devices 352 may be configured in various ways, such as for example, as a spray nozzle and/or a slot coating device. In some configurations, the adhesive applicator devices may be configured in accordance with the apparatuses and/or methods disclosed in U.S. Patent Nos. 8,186,296; 9,265,672; 9,248,054; and 9,295,590 and U.S. Patent Publication No. 2014/0148773 A1, which are all incorporated by reference herein.

As previously mentioned, the bonding station 304 may be configured as a mechanical bonding device that mechanically bonds the waist panels 158 with the primary carrier substrate 200. For example, the bonding station 304 in FIG. 4 may be configured that the first roll 342 and/or the second roll 344 may be configured as an anvil or pattern roll. In some configurations, a pattern roll may also comprise one or more bonding surfaces defined by bonding elements extending radially outward. As the pattern roll and the anvil roll rotate, the primary carrier substrate 200 and discrete waist panels 158 are advanced between the bonding surfaces and the pressing surface to mechanically bond or weld the primary carrier substrate 200 and waist panel 158 together to create the pressure bonds. It is also to be appreciated that the pattern roll and/or anvil roll may be configured to apply heat and pressure in various ways to perform mechanical bonding, such as for example, the mechanical bonding devices and methods disclosed in in U.S. Patent Nos. 4,854,984; 6,248,195; 8,778,127; 9,005,392; 9,962,297; and 10,052,237, which are all incorporated by reference herein.

It is to be appreciated that the bonding station 304 may be configured in various ways, such as with heated or unheated pattern rolls, anvil rolls and/or ultrasonic bonding devices. For example, the bonding station 304 may include a pattern roll and a pressing surface that comprises an energy transfer surface of an ultrasonic bonding device. As such, the bonding device may include a horn and may be configured to impart ultrasonic energy to the combined waist panel 158 and the primary carrier substrate 200. It is to be appreciated that aspects of the ultrasonic bonding device may be configured in various ways, such as for example linear or rotary type configurations, and such as disclosed for example in U.S. Patent Nos. 3,113,225; 3,562,041; 3,733,238; 5,110,403; 6,036,796; 6,508,641; and 6,645,330, which are all incorporated by reference herein. In some configurations, the ultrasonic bonding device may be configured as a linear oscillating type sonotrode, such as for example, available from Herrmann Ultrasonic, Inc. In some configurations, the sonotrode may include a plurality of sonotrodes nested together in the cross direction CD. It is also to be appreciated that rotary horns may also be configured to rotate at constant and/or variable angular velocities.

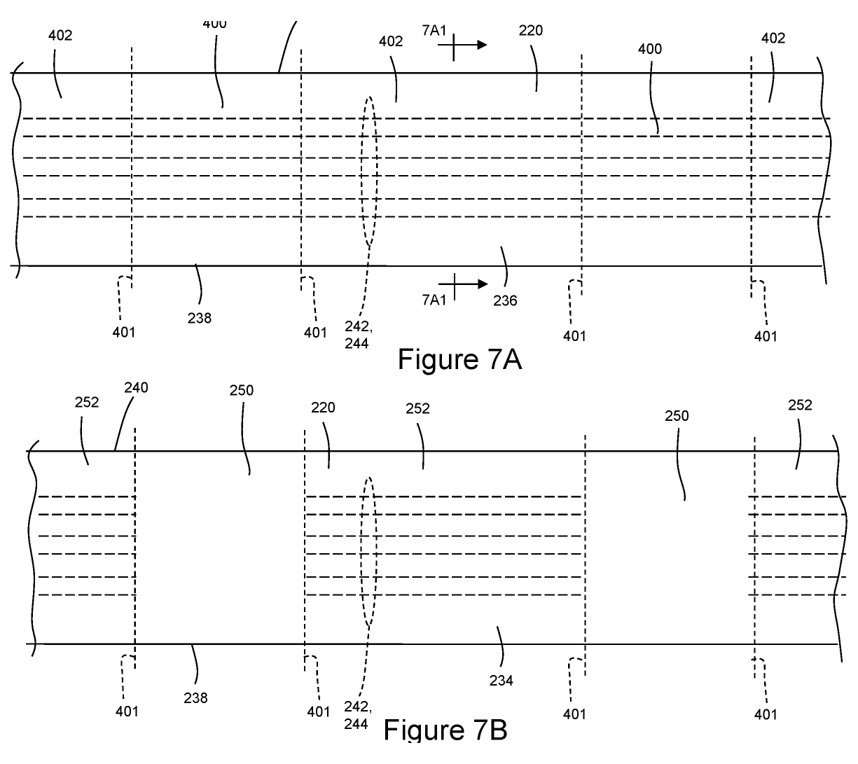

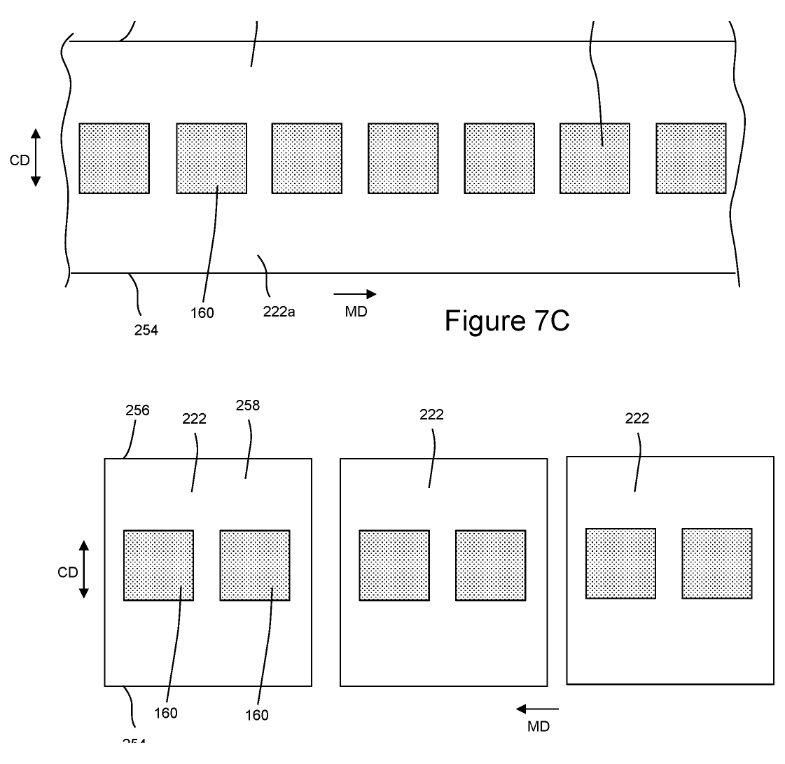

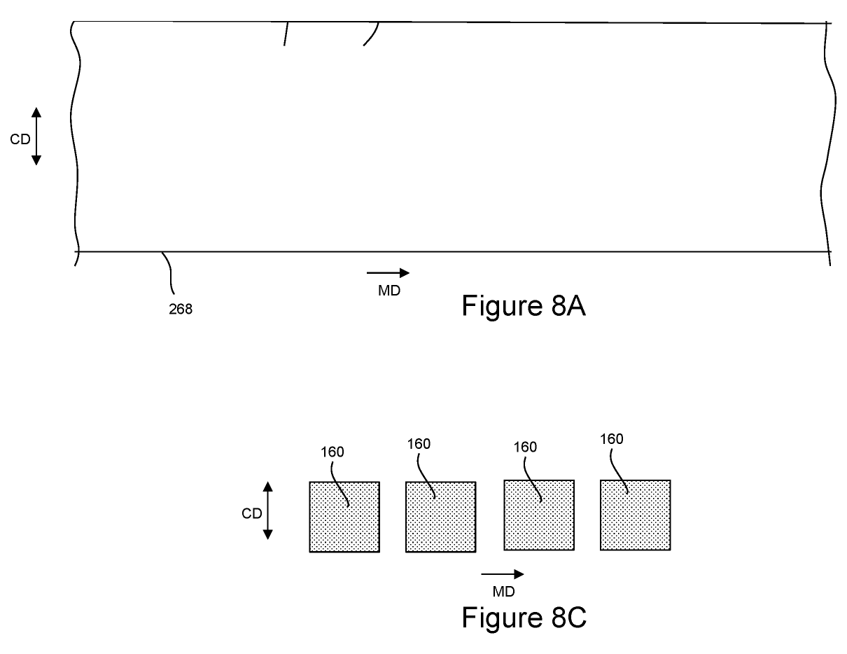

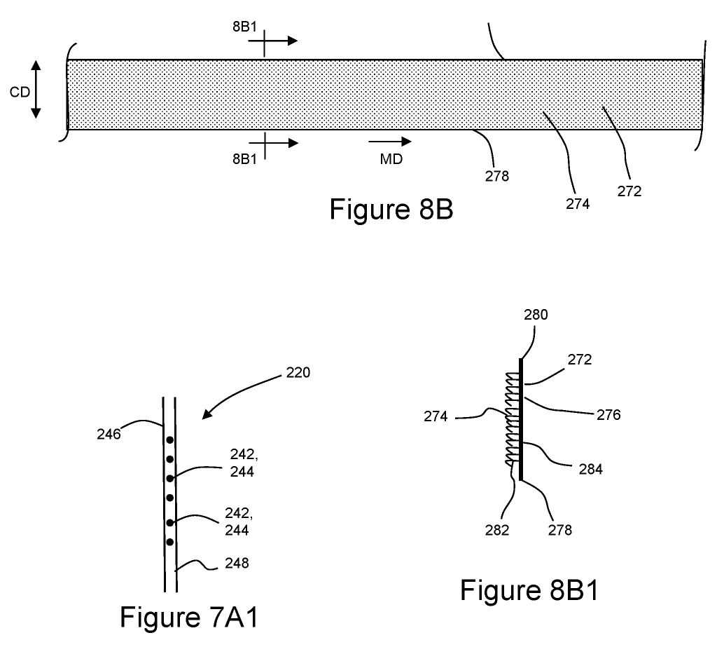

It is to be appreciated that the waist panels 158 herein may be configured in various ways, and as such, it is to be appreciated that the continuous waist panel laminate 158a from which the waist panels 158 are separated from may also be configured in various ways. In turn, the apparatus 300 shown in FIG. 4 may include assembly devices arranged in various ways to assemble such continuous waist panel laminates 158a. For example, as shown in FIGS. 4 and 7A, an elastic laminate 220 is advanced in a machine direction MD. As shown in FIG. 7A, the elastic laminate 220 may comprise a first surface 234 and an opposing second surface 236. The elastic laminate 220 further comprises a first edge 238 separated from a second edge 240 in a cross direction CD, wherein the elastic laminate 220 comprises a width defined by a distance extending in the cross direction CD between the first edge 238 and the second edge 240.

It is to be appreciated that the system 300 may include various apparatuses and utilize various methods to produce elastic laminates 220 that may be used to construct diaper components herein, such as waist panels 158. For example, the apparatus 300 shown in FIG. 4 may include a laminate converting apparatus 301 that is adapted to provide and/or manufacture elastic laminates 220. As discussed in more detail below, the elastic laminate 220 may be converted into a continuous waist panel material 158a and waist panels 158, such as discussed above. As shown in FIGS. 4, 7A, and 7A1, the elastic laminate 220 may comprise elastic material 242 interposed between a first substrate layer 246 and a second substrate layer 248. The apparatus 301 may stretch the elastic material 242 and join the stretched elastic material 242 with the first and second substrate layers 246, 248 to produce an elastomeric laminate 220. Although the elastic material 242 is illustrated and referred to herein as elastic strands 244, it is to be appreciated that in some configurations, elastic material 242 may include one or more continuous lengths of elastic strands, ribbons, and/or films. Elastic material 242 in the form of elastic strands 244 may extend in the machine direction MD. As such, a plurality of stretched elastic strands 244 may be positioned and bonded between the first substrate layer 246 and the second substrate layer 248. As such, the first surface 234 may be defined by the first substrate layer 246 and the second surface 236 may be defined by the second substrate layer 248.

It is to be appreciated that in some configurations, the first substrate layer 246 and the second substrate layer 248 herein may be defined by two discrete substrates or may be defined by folded portions of a single substrate. In some examples, the first and/or second substrate layers 246, 248 may include nonwovens and/or films. In addition, the elastic strands 244 may be configured in various ways and may have various decitex values. In some configurations, the elastic strands 244 may be configured with decitex values ranging from about 10 decitex to about 1000 decitex, specifically reciting all 1 decitex increments within the above-recited range and all ranges formed therein or thereby. It is also to be appreciated that the elastomeric laminates 220 assembled herein may include various quantities of elastic strands 244 spaced apart from each other by various distances and may include various decitex values. For example, the elastomeric laminates 220 herein may have various elastic densities, wherein the elastic density may be defined as decitex per elastomeric laminate width. For example, some elastomeric laminates 220 may have an elastic density from about 30 decitex/mm to about 150 decitex/mm, specifically reciting all 1 decitex/mm increments within the above-recited range and all ranges formed therein or thereby. In another example, the elastomeric laminates 220 herein may have various numbers of elastic strands 244 arranged in the cross direction CD per meter of elastomeric laminate cross directional width. For example, some elastomeric laminates 220 may have from about 500 elastic strands/meter of elastomeric laminate width to about 2000 elastic strands/meter of elastomeric laminate width, specifically reciting all 1 elastic strand/meter increments within the above-recited range and all ranges formed therein or thereby.

The elastic strands 244 may be intermittently bonded with the first substrate layer 246 and the second substrate layer 248 along the machine direction MD to form unbonded regions 400 where the elastic strands 244 are not bonded with the first substrate layer 246 and the second substrate layer 248. The unbonded regions 400 may be intermittently spaced along the machine directions between bonded regions 402 where the elastic strands 244 are bonded with the first substrate layer 246 and the second substrate layer 248. Perimeter shapes of the unbonded regions 400 are generically represented by dashed line borders 401 shown in FIG. 7A. It is to be appreciated that unbonded regions 400 may be configured with dashed lined border 401 that may define other shapes of the unbonded regions 400, such as for example, square, rectangular, circular, and elliptical.

With reference to FIGS. 4, 7A, and 7B, the elastic laminate 220 may advance to a cutting station 354 wherein one or more elastic strands 244 are severed in the unbonded region 400. As shown in FIG. 7B, the severed elastic strands 244 retract along the machine direction MD within the unbonded region 400 to form low-stretch zones 250. For example, the severed waist elastic strands 244 may retract in opposite directions from cut lines in the unbonded regions 400 and toward the bonded regions 402. In turn, the low-stretch zones 250 may be intermittently spaced from each other by high-stretch zones 252 along the machine direction MD. The low-stretch zones 250 define regions of the elastic laminate that have relatively less elasticity than the high-stretch zones 252. The elastic material 242 that has been cut and which are elastically contracted do not add any substantial amount of elastication to the low-stretch zone 250. As such, upon application of a force, the high-stretch zones 252 will elongate more than the low-stretch zones 250. In some configurations, the elastic laminate 220 may be configured with high-stretch zones 252 that are elastic and may be configured with low-stretch zones 250 that are not elastic or “inelastic.”

The elastic strands 244 may be severed once at the cutting station 354. However, it is to be appreciated that the cutting station 354 may be configured to sever the elastic strands 244 more than once at a plurality of cut lines. It is also to be appreciated that one or both of the first and second substrate layers 246, 248 of the elastic laminate 220 may be severed at the cut lines. And in some configurations, neither of the first and second substrate layers 246, 248 of the elastic laminate 220 are severed at the cut lines. It is also to be appreciated that the cutting station 354 may be adapted to sever various quantities of elastic strands 244.

It is to be appreciated that the cutting station 354 may be configured in various ways. For example, the cutting station 354 may comprise various arrangements of a knife roll with blades and an anvil roll, such as disclosed in PCT Publication No. WO2023/088179 A1; U.S. Patent Nos. 8,440,043; 10,759,153; and 10,807,263; and U.S. Patent Publication Nos. 2020/0180182 A1 and 2024/0000619 A1, which are incorporated by reference herein. As such, the elastic laminate 220 may advance between a knife roll and anvil roll to sever the elastic strands 244 at the cutting station 354. As opposed to or in addition to blades, it is to be appreciated that the cutting station 354 may be configured to perform cutting operations in various other ways, such as with lasers or ultrasonics, for example as disclosed in U.S. Patent Publication Nos. 2016/0354254 A1; 2016/0128874 A1; 2017/0266941 A1; 2017/0266057 A1; and 2017/0266056 A1, which are all incorporated by reference herein.

As shown in FIGS. 4 and 7B-7D, the elastic laminate 220 may advance from the cutting station 354 to a first disposition station 356 that may be configured to combine fastener panels 222 with the elastic laminate 220. For example, as shown in FIGS. 4 and 7C, a continuous length of fastener panels 222a may advance to the first disposition station 356. The continuous length of fastener panels 222a may comprise a first longitudinal edge 254 and a second longitudinal edge 256 separated from each other in a cross direction CD. The continuous length of fastener panels 222a may also comprise a first surface 258 and an opposing second surface 260, wherein fastener components 160 may be positioned on the first surface 258a. The first disposition station 356 may be adapted to separate discrete fastener panels 222 from the continuous length of fastener panels 222a, such as shown in FIG. 7D. As such, the fastener panel 222 also includes first and second longitudinal edges 254, 256 that correspond with the longitudinal edges 254, 256 of the continuous length of fastener panels 222a. The fastener panels 222 also each include first and second surfaces 258, 260 that correspond with the first and second surfaces 258, 260 of the continuous length of fastener panels 222a.

The first disposition station 356 may operate to position the second surfaces 260 of the fastener panels 222 in a facing relationship with the first surface 234 of the elastic laminate 220 with the fastener panels 222 separated from each other along the machine direction MD to form the waist panel laminate 158a, such as shown in FIG. 6A. The first disposition station 356 may also be configured to bond the fastener panels 222 with the elastic laminate 220. It is to be appreciated that the first disposition station 356 may be configured to bond the fastener panels 222 with the elastic laminate 220 in various ways, such as with adhesive, mechanical bonding, and combinations of thereof.

It is also to be appreciated that the first disposition station 356 may be configured in various ways. For example, the first disposition station 356 may comprise various methods and apparatuses for separating discrete pieces (such as fastener panels 222) from a first continuous web (such as the continuous length of fastener panels 222a) and applying the discrete pieces to a second continuous web (such as the elastic laminate 220), such as disclosed for example in U.S. Patent Nos. 5,415,716; 5,702,551; 6,475,325, and 6,705,453, which are incorporated by reference herein. For example, the first disposition station may comprise a “cut and slip apparatus,” also referred to as a “slip and cut apparatus, to apply discrete pieces of a first web to a second continuous web. In some examples, the cut and slip apparatus may comprise a rotating anvil roll and a rotating knife roll. In some configurations, a first web may advance at a relatively low speed along a surface of the rotating anvil, which is moving at a relatively higher surface speed and upon which the first web is allowed to “slip.” A knife-edge on the rotating knife roll, cuts a discrete piece from the first web against the anvil roll. Once cut, the discrete piece may be held by vacuum forces on the anvil roll as the discrete piece is advanced at the speed of the anvil roll downstream to where the discrete piece is transferred to the second web.

In combination with or instead of combining discrete fastener panels 222 with the elastic laminate 220, the first disposition station 356 may be configured to integrally form fasteners 160, such as hooks, directly from material of the elastic laminate 220, such as disclosed for example in U.S. Patent Nos. 6,478,784; 8,784,722; 11,376,170; and 11,576,822; and U.S. Patent Publication No. 2018/0141266 A1, which are all incorporated herein by reference.

It is to be appreciated that the fastener panels 222 herein may be configured in various ways, and as such, it is to be appreciated that the continuous length of fastener panels 222a from which the discrete fastener panels 222 are separated from may also be configured in various ways. In turn, the apparatus 300 shown in FIG. 4 may include assembly systems arranged in various ways to assemble such continuous lengths of fastener panels 222a. For example, as shown in FIGS. 4 and 8A, a secondary carrier substrate 262 is advanced in a machine direction MD to a second disposition station 358 that may be configured to combine fasteners 160 with the secondary carrier substrate 262.

As shown in FIG. 8A, the secondary carrier substrate 262 may comprise a first surface 264 and an opposing second surface 266. The secondary carrier substrate 262 further comprises a first edge 268 separated from a second edge 270 in a cross direction CD, wherein the secondary carrier substrate 262 comprises a width defined by a distance extending in the cross direction CD between the first edge 268 and the second edge 270. As shown in FIGS. 4 and 8B, a continuous length of fastener material 272 may advance to the second disposition station 358. As shown in FIG. 8B, the continuous length of fastener material 272 may comprise a first surface 274 and an opposing second surface 276. The continuous length of fastener material 272 further comprises a first edge 278 separated from a second edge 280 in a cross direction CD, wherein the continuous length of fastener material 272 comprises a width defined by a distance extending in the cross direction CD between the first edge 278 and the second edge 280.

As previously discussed, it is to be appreciated that the fasteners 160 herein, as well as the continuous length of fastener material 272, may be configured in various ways, such as for example, mechanical fasteners in the form of hook fasteners, loop fasteners, macrofasteners, buttons, snaps, tab and slot fasteners, tape fasteners, adhesive fasteners, cohesive fasteners, magnetic fasteners, hermaphroditic fasteners, and the like. For example, as shown in FIGS. 8B and 8B1, the continuous length of fastener material 272 may comprise hooks 282 protruding from a base 284. As such, the first surface 274 of the continuous length of fastener material 272 may be defined by the hooks 282 and the base 284, and the second surface of the continuous length of fastener material 272 may be defined by the base 284. It is also to be appreciated that the base 284 may be configured in various ways. For example, the base 284 may comprise a thermoplastic film. In some configurations, the base 284 may comprise a laminate with various layers bonded together. It is also to be appreciated that such layers may be bonded together in various ways, such as with adhesive, mechanical bonding, and/or extrusion bonding. In some configurations, layers of the base 284 may be bonded together with extrusion or melt type bonding such as disclosed for example in U.S. Patent Publication No. 2021/0045931 A1. For example, the base 284 may comprise a thermoplastic film layer and a nonwoven backing layer, wherein the thermoplastic film layer is bonded with the nonwoven backing layer with extrusion bonds.

With continued reference to FIGS. 4, 6A, and 8A-8C, the second disposition station may be adapted to separate discrete fasteners 160 from the continuous length of fastener material 272. As such, the fasteners 160 also include first and second longitudinal edges 278, 280 that correspond with the longitudinal edges 278, 280 of the continuous fastener material 272. The fasteners 160 also each include first and second surfaces 274, 276 that correspond with the first and second surfaces 274, 276 of the continuous length of fastener material 272. The second disposition station 358 may operate to position the second surfaces 276 of the fasteners 160 in a facing relationship with the first surface 264 of the secondary carrier substrate 262 with the fasteners 160 separated from each other along the machine direction MD to form the continuous length of fastener panels 222a, such as shown in FIG. 7C. The second disposition station 358 may also be configured to bond the fasteners 160 with the secondary carrier substrate 262. It is to be appreciated that the second disposition station 358 may be configured to bond the fasteners 160 with the secondary carrier substrate 262 in various ways, such as with adhesive, mechanical bonding, and combinations of thereof.

It is also to be appreciated that the second disposition station 358 may be configured in various ways. For example, the second disposition station 358 may comprise various methods and apparatuses for separating discrete pieces (such as fasteners 160) from a first continuous web (such as the continuous length of fastener material 272) and applying the discrete pieces to a second continuous web (such as the secondary carrier substrate 262), such as disclosed for example in U.S. Patent Nos. 5,415,716; 5,702,551; 6,475,325, and 6,705,453, which are incorporated by reference herein. In combination with or instead of combining discrete fasteners 160 with the secondary carrier substrate 262, the second disposition station 358 may be configured to integrally form fasteners 160, such as hooks, directly from material of the elastic laminate, such as disclosed for example in U.S. Patent Nos. 6,478,784; 8,784,722; 11,376,170; and 11,576,822; and U.S. Patent Publication No. 2018/0141266 A1, which are all incorporated herein by reference. In some configurations, the second disposition station 358 may comprise a “cut and slip apparatus” such as described above, also referred to as a “slip and cut apparatus,” to apply discrete pieces of a first web to a second continuous web. It is also to be appreciated that the apparatus 300 may comprise more than one second disposition station 358 for application of fasteners 160.

Although the apparatus 300 is illustrated and described as being configured to assemble a continuous waist panel laminate 158a as a continuous part of an absorbent article assembly process, it is to be appreciated that the continuous waist panel laminate 158a may be assembled in a separate process. In turn, the continuous waist panel laminate 158a may be accumulated, such as for example, by being wound onto a roll or being festooned in a container. The accumulated the continuous waist panel laminate 158a may be stored and/or moved to a location for incorporation into a manufacturing process, such as an absorbent article assembly process, wherein the continuous waist panel laminate 158a may be converted into an absorbent article component.

### Combinations

A1. A method for assembling absorbent articles, the method comprising steps of: providing a secondary carrier substrate comprising fasteners intermittently spaced apart from each other along a length of the secondary carrier substrate; separating the secondary carrier substrate into discrete fastener panels, each fastener panel comprising a first surface and an opposing second surface, wherein at least one fastener is positioned on the first surface; providing an elastic laminate comprising a first surface and an opposing second surface; bonding the second surfaces of the fastener panels with the first surface of the elastic laminate to form a waist panel laminate, the fastener panels being intermittently spaced from each other along a length of the elastic laminate; separating the waist panel laminate along at least one line of separation extending through the elastic laminate and the fastener panels to form discrete waist panels, each discrete waist panel comprising a first surface and an opposing second surface, a first end region, a second end region, and central region separating the first end region from the second end region, wherein each waist panel comprises a first portion of a first separated fastener panel on the first surface of the first end region and a second portion of a second separated fastener panel on the first surface of the second end region; advancing a primary carrier substrate in a machine direction, the primary carrier substrate comprising a first edge separated from a second edge in a cross direction, wherein first and second edges of the primary carrier substrate are contoured to define relatively narrow regions arranged intermittently between relatively wide regions along the machine direction, wherein the relatively wide regions comprise integrally formed wing panels; and bonding the waist panels with the primary carrier substrate, wherein the waist panels extend in a cross direction such that the first end region extends outboard from the first edge and the second end region extends outboard from the second edge of the primary carrier substrate.

A2. The method of paragraph A1, wherein the elastic laminate comprises low stretch zones arranged intermittently between high stretch zones along the machine direction.

A3. The method of paragraph A1 or A2, wherein step of providing the elastic laminate further comprises bonding stretched elastic strands with a nonwoven substrate.

A4. The method of paragraph A3, further comprising a step of severing the elastic strands to form the low stretch zones.

A5. The method of paragraph A4, wherein the elastic strands are not bonded with the nonwoven substrate in unbonded regions, and wherein the elastic strands are bonded with the nonwoven substrate in bonded regions, the bonded regions intermittently arranged between the unbonded regions, and further comprising a step of severing the elastic strands in the unbonded regions to form the low stretch zones.

A6. The method of paragraph A2, wherein the step of bonding second surfaces of the fastener panels with the first surface of the elastic laminate further comprises bonding the fastener panels with the low stretch zones of the elastic laminate.

A7. The method of any of paragraphs A1 to A6, wherein the step of separating the secondary carrier substrate into discrete fastener panels further comprises cutting the secondary carrier substrate.

A8. The method of paragraph A7, wherein the step of cutting the secondary carrier substrate further comprises cutting through a fastener to form a first fastener and a second fastener.

A9. The method of paragraph A7, wherein the at least one fastener positioned on the first surface of the carrier substrate comprises a first fastener and a second fastener, and wherein the step of cutting the secondary carrier substrate further comprises cutting between the first fastener and the second fastener.

A10. The method of any of paragraphs A1 to A9, wherein the step of providing the secondary carrier substrate further comprises bonding discrete fasteners with the secondary carrier substrate.

A11. The method of any of paragraphs A1 to A9, wherein the step of providing the secondary carrier substrate further comprises forming discrete fasteners from material of the secondary carrier substrate.

A12. The method of any of paragraphs A1 to A11, wherein the primary carrier substrate comprises a continuous absorbent chassis comprising a continuous backsheet substrate bonded with a continuous topsheet substrate with an absorbent core positioned between the continuous backsheet and the continuous topsheet.

A13. The method of paragraph A12, further comprising a step of trimming areas of both the continuous backsheet substrate and the continuous topsheet substrate along the machine direction to form the relatively wide regions and the relatively narrow regions.

A14. The method of paragraph A12, further comprising a step of cutting in the cross direction through the relatively wide regions of the continuous absorbent chassis to form discrete absorbent articles.

A15. The method of paragraph A12, further comprising a step of bonding leg cuffs with the backsheet or the topsheet.

A16. The method of any of paragraphs A1 to A11, wherein the primary carrier substrate comprises a continuous topsheet substrate comprising a first surface and an opposing second surface, and further comprising a step of bonding the second surfaces of the waist panels with the first surface of the continuous topsheet substrate.

A17. The method of paragraph A16, wherein the step of bonding the second surfaces of the waist panels with the first surface of the continuous topsheet substrate further comprises bonding a first region of each waist panel with the continuous topsheet substrate, wherein a second region of each waist panel remains unattached to the continuous topsheet substrate to define a pocket between the waist panel and the continuous topsheet substrate.

A18. The method of paragraph A17, wherein each waist panel comprises a first lateral edge and a second lateral edge separated from the first lateral edge in the machine direction, and wherein the first lateral edge is not bonded with the topsheet substrate to define an opening of the pocket.

A19. The method of paragraph A18, wherein the first lateral edge is a trailing edge and the second lateral edge is a leading edge.

A20. The method of paragraph A16, further comprising a step of bonding a continuous backsheet substrate with the second surface of the continuous topsheet.

A21. The method of any of paragraphs A1 to A11, wherein the primary carrier substrate comprises a continuous backsheet substrate comprising a first surface and an opposing second surface, and further comprising a step of bonding the first surfaces of the waist panels with the second surface of the continuous backsheet substrate.

A22. The method of paragraph A21, further comprising a step of bonding a continuous topsheet substrate with the first surface of the continuous backsheet.

A23. The method of any of paragraphs A1 to A22, wherein the step of separating the waist panel laminate along at least one line of separation further comprises cutting a trim piece from the waist panel laminate wherein the trim piece extends between a first line of separation and a second line of separation.

A24. The method of any of paragraphs A1 to A22, wherein the at least one line of separation comprises a line of weakness.

A25. The method of paragraph A24, wherein the step of separating the waist panel laminate along the at least one line of separation further comprises tearing the waist panel laminate along the line of weakness.

A26. The method of any of paragraphs A1 to A25, further comprising pivoting the discrete waist panels by about 90 degrees before bonding the waist panels with the primary carrier substrate.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260^{TM}, SHE150^{TM}, or SGM9450F^{TM} (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on June 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling absorbent articles, the method comprising steps of:
providing a secondary carrier substrate comprising fasteners intermittently spaced apart from each other along a length of the secondary carrier substrate;
separating the secondary carrier substrate into discrete fastener panels, each fastener panel comprising a first surface and an opposing second surface, wherein at least one fastener is positioned on the first surface;
providing an elastic laminate comprising a first surface and an opposing second surface;
bonding the second surfaces of the fastener panels with the first surface of the elastic laminate to form a waist panel laminate, the fastener panels being intermittently spaced from each other along a length of the elastic laminate;
separating the waist panel laminate along at least one line of separation extending through the elastic laminate and the fastener panels to form discrete waist panels, each discrete waist panel comprising a first surface and an opposing second surface, a first end region, a second end region, and central region separating the first end region from the second end region, wherein each waist panel comprises a first portion of a first separated fastener panel on the first surface of the first end region and a second portion of a second separated fastener panel on the first surface of the second end region;
advancing a primary carrier substrate in a machine direction, the primary carrier substrate comprising a first edge separated from a second edge in a cross direction, wherein first and second edges of the primary carrier substrate are contoured to define relatively narrow regions arranged intermittently between relatively wide regions along the machine direction, wherein the relatively wide regions comprise integrally formed wing panels; and
bonding the waist panels with the primary carrier substrate, wherein the waist panels extend in a cross direction such that the first end region extends outboard from the first edge and the second end region extends outboard from the second edge of the primary carrier substrate.

2. The method of claim 1, wherein the elastic laminate comprises low stretch zones arranged intermittently between high stretch zones along the machine direction.

3. The method of claim 2, wherein step of providing the elastic laminate further comprises bonding stretched elastic strands with a nonwoven substrate.

4. The method of claim 3, further comprising a step of severing the elastic strands to form the low stretch zones.

5. The method of claim 4, wherein the elastic strands are not bonded with the nonwoven substrate in unbonded regions, and wherein the elastic strands are bonded with the nonwoven substrate in bonded regions, the bonded regions intermittently arranged between the unbonded regions, and further comprising a step of severing the elastic strands in the unbonded regions to form the low stretch zones.

6. The method of claim 2, wherein the step of bonding second surfaces of the fastener panels with the first surface of the elastic laminate further comprises bonding the fastener panels with the low stretch zones of the elastic laminate.

7. The method of claim 1, wherein the step of separating the secondary carrier substrate into discrete fastener panels further comprises cutting the secondary carrier substrate.

8. The method of claim 7, wherein the step of cutting the secondary carrier substrate further comprises cutting through a fastener to form a first fastener and a second fastener.

9. The method of claim 7, wherein the at least one fastener positioned on the first surface of the carrier substrate comprises a first fastener and a second fastener, and wherein the step of cutting the secondary carrier substrate further comprises cutting between the first fastener and the second fastener.

10. The method of claim 1, wherein the step of providing the secondary carrier substrate further comprises bonding discrete fasteners with the secondary carrier substrate.

11. The method of claim 1, wherein the step of providing the secondary carrier substrate further comprises forming discrete fasteners from material of the secondary carrier substrate.

12. The method of claim 1, wherein the primary carrier substrate comprises a continuous absorbent chassis comprising a continuous backsheet substrate bonded with a continuous topsheet substrate with an absorbent core positioned between the continuous backsheet and the continuous topsheet.

13. The method of claim 12, further comprising a step of trimming areas of both the continuous backsheet substrate and the continuous topsheet substrate along the machine direction to form the relatively wide regions and the relatively narrow regions.

14. The method of claim 12, further comprising a step of cutting in the cross direction through the relatively wide regions of the continuous absorbent chassis to form discrete absorbent articles.

15. The method of claim 12, further comprising a step of bonding leg cuffs with the backsheet or the topsheet.

16. The method of claim 1, wherein the primary carrier substrate comprises a continuous topsheet substrate comprising a first surface and an opposing second surface, and further comprising a step of bonding the second surfaces of the waist panels with the first surface of the continuous topsheet substrate.

17. The method of claim 16, wherein the step of bonding the second surfaces of the waist panels with the first surface of the continuous topsheet substrate further comprises bonding a first region of each waist panel with the continuous topsheet substrate, wherein a second region of each waist panel remains unattached to the continuous topsheet substrate to define a pocket between the waist panel and the continuous topsheet substrate.

18. The method of claim 17, wherein each waist panel comprises a first lateral edge and a second lateral edge separated from the first lateral edge in the machine direction, and wherein the first lateral edge is not bonded with the topsheet substrate to define an opening of the pocket.

19. The method of claim 18, wherein the first lateral edge is a trailing edge and the second lateral edge is a leading edge.

20. The method of claim 16, further comprising a step of bonding a continuous backsheet substrate with the second surface of the continuous topsheet.
